SEQUENCE LISTING (101 sequences)

SEQ ID NO: 1 - protein, 14 aa, Artificial Sequence
  artificial antibody sequence
TGSSSNIGAGYDVH

SEQ ID NO: 2 - protein, 7 aa, Artificial Sequence
  artificial antibody sequence
ENDRRPS

SEQ ID NO: 3 - protein, 11 aa, Artificial Sequence
  artificial antibody sequence
AAWDDSLNGPL

SEQ ID NO: 4 - protein, 111 aa, Artificial Sequence
  artificial antibody sequence
QSVLTQPPSASGTPGQRVTISCTGSSSNIGAGYDVHWYQQLPGTAPKLLIYENDRRPSGVPDRFSGSKSGTSASLAISGLRSEDEADYYCAAWDDSLNGPLFGGGTKLTVL

SEQ ID NO: 5 - protein, 5 aa, Artificial Sequence
  artificial antibody sequence
NYAMS

SEQ ID NO: 6 - protein, 17 aa, Artificial Sequence
  artificial antibody sequence
AISGSGGSTFYADSVKG

SEQ ID NO: 7 - protein, 8 aa, Artificial Sequence
  artificial antibody sequence
DGTDAFDI

SEQ ID NO: 8 - protein, 117 aa, Artificial Sequence
  artificial antibody sequence
EVQLLESGGGLVQPGGSLRLSCAASGFTFSNYAMSWVRQAPGKGLEWVSAISGSGGSTFYADSVKGRFTISRDNSKNTLYLQMNSLRAEDTAVYYCATDGTDAFDIWGQGTLVTVSS

SEQ ID NO: 9 - protein, 14 aa, Artificial Sequence
  artificial antibody sequence
TGSSSNIGAGYVVH

SEQ ID NO: 10 - protein, 7 aa, Artificial Sequence
  artificial antibody sequence
NNSQRPP

SEQ ID NO: 11 - protein, 11 aa, Artificial Sequence
  artificial antibody sequence
ASWDDSLSGVV

SEQ ID NO: 12 - protein, 111 aa, Artificial Sequence
  artificial antibody sequence
QSVLTQPPSASGTPGQRVTISCTGSSSNIGAGYVVHWYQQLPGTAPKLLIYNNSQRPPGVPDRFSGSKSGTSASLAISGLRSEDEADYYCASWDDSLSGVVFGGGTKLTVL

SEQ ID NO: 13 - protein, 5 aa, Artificial Sequence
  artificial antibody sequence
SYAMS

SEQ ID NO: 14 - protein, 17 aa, Artificial Sequence
  artificial antibody sequence
AISGSGGSTYYADSVKG

SEQ ID NO: 15 - protein, 12 aa, Artificial Sequence
  artificial antibody sequence
EQWLGAEGAFDI

SEQ ID NO: 16 - protein, 121 aa, Artificial Sequence
  artificial antibody sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCATEQWLGAEGAFDIWGQGTLVTVSS

SEQ ID NO: 17 - protein, 12 aa, Artificial Sequence
  artificial antibody sequence
SGSSNIGNNAVN

SEQ ID NO: 18 - protein, 7 aa, Artificial Sequence
  artificial antibody sequence
GNSNRPS

SEQ ID NO: 19 - protein, 10 aa, Artificial Sequence
  artificial antibody sequence
QSYDSSLSGV

SEQ ID NO: 20 - protein, 109 aa, Artificial Sequence
  artificial antibody sequence
QSVLTQPPSASGTPGQRVTISCSGSSSNIGNNAVNWYQQLPGTAPKLLIYGNSNRPSGVPDRFSGSKSGTSASLAISGLRSEDEADYYCQSYDSSLSGVFGGGTKLTVL

SEQ ID NO: 21 - protein, 5 aa, Artificial Sequence
  artificial antibody sequence
SYAMS

SEQ ID NO: 22 - protein, 17 aa, Artificial Sequence
  artificial antibody sequence
GVSWNGSRTHYADSVKG

SEQ ID NO: 23 - protein, 10 aa, Artificial Sequence
  artificial antibody sequence
ERLGKWYFDL

SEQ ID NO: 24 - protein, 119 aa, Artificial Sequence
  artificial antibody sequence
EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARERLGKWYFDLWGRGTLVTVSS

SEQ ID NO: 25 - protein, 586 aa, Artificial Sequence
  artificial antibody sequence
MYGFVNHALELLVIRNYGPEVWEDIKKEAQLDEEGQFLVRIIYDDSKTYDLVAAASKVLNLNAGEILQMFGKMFFVFCQESGYDTILRVLGSNVREFLQNLDALHDHLATIYPGMRAPSFRCTDAEKGKGLILHYYSEREGLQDIVIGIIKTVAQQIHGTEIDMKVIQQRNEECDHTQFLIEEKESKEEDFYEDLDRFEENGTQESRISPYTFCKAFPFHIIFDRDLVVTQCGNAIYRVLPQLQPGNCSLLSVFSLVRPHIDISFHGILSHINTVFVLRSKEGLLDVEKLECEDELTGTEISCLRLKGQMIYLPEADSILFLCSPSVMNLDDLTRRGLYLSDIPLHDATRDLVLLGEQFREEYKLTQELEILTDRLQLTLRALEDEKKKTDTGIVGFNAFCSKHASGEGAMKIVNLLNDLYTRFDTLTDSRKNPFVYKVETVGDKYMTVSGLPEPCIHHARSICHLALDMMEIAGQVQVDGESVQITIGIHTGEVVTGVIGQRMPRYCLFGNTVNLTSRTETTGEKGKINVSEYTYRCLMSPENSDPQFHLEHRGPVSMKGKKEPMQVWFLSRKNTGTEETKQDDD

SEQ ID NO: 26 - protein, 217 aa, Artificial Sequence
  artificial antibody sequence
QSVLTQPPSASGTPGQRVTISCTGSSSNIGAGYDVHWYQQLPGTAPKLLIYENDRRPSGVPDRFSGSKSGTSASLAISGLRSEDEADYYCAAWDDSLNGPLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - protein, 446 aa, Artificial Sequence
  artificial antibody sequence
EVQLLESGGGLVQPGGSLRLSCAASGFTFSNYAMSWVRQAPGKGLEWVSAISGSGGSTFYADSVKGRFTISRDNSKNTLYLQMNSLRAEDTAVYYCATDGTDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 217 aa, Artificial Sequence
  artificial antibody sequence
QSVLTQPPSASGTPGQRVTISCTGSSSNIGAGYVVHWYQQLPGTAPKLLIYNNSQRPPGVPDRFSGSKSGTSASLAISGLRSEDEADYYCASWDDSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 450 aa, Artificial Sequence
  artificial antibody sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCATEQWLGAEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 215 aa, Artificial Sequence
  artificial antibody sequence
QSVLTQPPSASGTPGQRVTISCSGSSSNIGNNAVNWYQQLPGTAPKLLIYGNSNRPSGVPDRFSGSKSGTSASLAISGLRSEDEADYYCQSYDSSLSGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 448 aa, Artificial Sequence
  artificial antibody sequence
EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARERLGKWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 13 aa, Artificial Sequence
  artificial antibody sequence
SGSSSNIGSNYVY

SEQ ID NO: 33 - protein, 7 aa, Artificial Sequence
  artificial antibody sequence
RNNQRPS

SEQ ID NO: 34 - protein, 11 aa, Artificial Sequence
  artificial antibody sequence
TAWDDSLSAVV

SEQ ID NO: 35 - protein, 110 aa, Artificial Sequence
  artificial antibody sequence
QSVLTQPPSASGTPGQRVTISCSGSSSNIGSNYVYWYQQLPGTAPKLLIYRNNQRPSGVPDRFSGSKSGTSASLAISGLRSEDEADYYCTAWDDSLSAVVFGGGTKLTVL

SEQ ID NO: 36 - protein, 5 aa, Artificial Sequence
  artificial antibody sequence
NYVMS

SEQ ID NO: 37 - protein, 17 aa, Artificial Sequence
  artificial antibody sequence
GVSWNGSRTHYVDSVKR

SEQ ID NO: 38 - protein, 10 aa, Artificial Sequence
  artificial antibody sequence
GLRYSSPFDF

SEQ ID NO: 39 - protein, 119 aa, Artificial Sequence
  artificial antibody sequence
EVQLLESGGGLVQPGGSLRLSCAASGFTFSNYVMSWVRQAPGKGLEWVSGVSWNGSRTHYVDSVKRRFTISRDNSKNTLYLQMNSLRAEDTAVYYCARGLRYSSPFDFWGQGTLVTVSS

SEQ ID NO: 40 - protein, 13 aa, Artificial Sequence
  artificial antibody sequence
SGSSSNIGSNTVN

SEQ ID NO: 41 - protein, 7 aa, Artificial Sequence
  artificial antibody sequence
GNSNRPS

SEQ ID NO: 42 - protein, 11 aa, Artificial Sequence
  artificial antibody sequence
AVWDDSLNGWV

SEQ ID NO: 43 - protein, 110 aa, Artificial Sequence
  artificial antibody sequence
QSVLTQPPSASGTPGQRVTISCSGSSSNIGSNTVNWYQQLPGTAPKLLIYGNSNRPSGVPDRFSGSKSGTSASLAISGLRSEDEADYYCAVWDDSLNGWVFGGGTKLTVL

SEQ ID NO: 44 - protein, 5 aa, Artificial Sequence
  artificial antibody sequence
RYGIH

SEQ ID NO: 45 - protein, 17 aa, Artificial Sequence
  artificial antibody sequence
VISYDGTNKYYADSVKG

SEQ ID NO: 46 - protein, 13 aa, Artificial Sequence
  artificial antibody sequence
ARSRWASLGAFDI

SEQ ID NO: 47 - protein, 122 aa, Artificial Sequence
  artificial antibody sequence
EVQLLESGGGLVQPGGSLRLSCAASGFTFSRYGIHWVRQAPGKGLEWVAVISYDGTNKYYADSVKGRFTISRDNSKNTLYLQMNSLRAEDTAVYYCARARSRWASLGAFDIWGQGTLVTVSS

SEQ ID NO: 48 - protein, 13 aa, Artificial Sequence
  artificial antibody sequence
SGSGSNIGNNAVN

SEQ ID NO: 49 - protein, 7 aa, Artificial Sequence
  artificial antibody sequence
GNSNRPS

SEQ ID NO: 50 - protein, 13 aa, Artificial Sequence
  artificial antibody sequence
QSYGTSLSGSRVL

SEQ ID NO: 51 - protein, 112 aa, Artificial Sequence
  artificial antibody sequence
QSVLTQPPSASGTPGQRVTISCSGSGSNIGNNAVNWYQQLPGTAPKLLIYGNSNRPSGVPDRFSGSKSGTSASLAISGLRSEDEADYYCQSYGTSLSGSRVLFGGGTKLTVL

SEQ ID NO: 52 - protein, 5 aa, Artificial Sequence
  artificial antibody sequence
KYWMH

SEQ ID NO: 53 - protein, 17 aa, Artificial Sequence
  artificial antibody sequence
SVSASGGSIYYADSVRG

SEQ ID NO: 54 - protein, 16 aa, Artificial Sequence
  artificial antibody sequence
GPFWSGYYRLDGLVDY

SEQ ID NO: 55 - protein, 125 aa, Artificial Sequence
  artificial antibody sequence
EVQLLESGGGLVQPGGSLRLSCAASGFTFRKYWMHWVRQTPGKGLEWVSSVSASGGSIYYADSVRGRFTISRDNSKNTLYLQMNSLRAEDTAVYYCARGPFWSGYYRLDGLVDYWGQGTLVTVSS

SEQ ID NO: 56 - protein, 13 aa, Artificial Sequence
  artificial antibody sequence
SGSSSNIGNNAVN

SEQ ID NO: 57 - protein, 7 aa, Artificial Sequence
  artificial antibody sequence
RDDRLPS

SEQ ID NO: 58 - protein, 10 aa, Artificial Sequence
  artificial antibody sequence
SSYTTSSTVV

SEQ ID NO: 59 - protein, 109 aa, Artificial Sequence
  artificial antibody sequence
QSVLTQPPSASGTPGQRVTISCSGSSSNIGNNAVNWYQQLPGTAPKLLIYRDDRLPSGVPDRFSGSKSGTSASLAISGLRSEDEADYYCSSYTTSSTVVFGGGTKLTVL

SEQ ID NO: 60 - protein, 5 aa, Artificial Sequence
  artificial antibody sequence
RYAMS

SEQ ID NO: 61 - protein, 17 aa, Artificial Sequence
  artificial antibody sequence
GVSWNGSRTHYVGSVKR

SEQ ID NO: 62 - protein, 10 aa, Artificial Sequence
  artificial antibody sequence
ERLGKWYFDL

SEQ ID NO: 63 - protein, 119 aa, Artificial Sequence
  artificial antibody sequence
EVQLLESGGGLVQPGGSLRLSCAASGFTFSRYAMSWVRQAPGKGLEWVSGVSWNGSRTHYVGSVKRRFTISRDNSKNTLYLQMNSLRAEDTAVYYCARERLGKWYFDLWGQGTLVTVSS

SEQ ID NO: 64 - protein, 13 aa, Artificial Sequence
  artificial antibody sequence
SGSRSNIGSSVVS

SEQ ID NO: 65 - protein, 7 aa, Artificial Sequence
  artificial antibody sequence
GNNQRPS

SEQ ID NO: 66 - protein, 10 aa, Artificial Sequence
  artificial antibody sequence
TSYAGSNNLV

SEQ ID NO: 67 - protein, 109 aa, Artificial Sequence
  artificial antibody sequence
QSVLTQPPSASGTPGQRVTISCSGSRSNIGSSVVSWYQQLPGTAPKLLIYGNNQRPSGVPDRFSGSKSGTSASLAISGLRSEDEADYYCTSYAGSNNLVFGGGTKLTVL

SEQ ID NO: 68 - protein, 5 aa, Artificial Sequence
  artificial antibody sequence
SYSMN

SEQ ID NO: 69 - protein, 17 aa, Artificial Sequence
  artificial antibody sequence
YISRSSGAIYYADSVKG

SEQ ID NO: 70 - protein, 10 aa, Artificial Sequence
  artificial antibody sequence
ERLGKWYFDL

SEQ ID NO: 71 - protein, 119 aa, Artificial Sequence
  artificial antibody sequence
EVQLLESGGGLVQPGGSLRLSCAASGFTFSSYSMNWVRQAPGKGLEWVSYISRSSGAIYYADSVKGRFTISRDNSKNTLYLQMNSLRAEDTAVYYCARERLGKWYFDLWGQGTLVTVSS

SEQ ID NO: 72 - protein, 14 aa, Artificial Sequence
  artificial antibody sequence
TGSSSNIGAGYDVH

SEQ ID NO: 73 - protein, 7 aa, Artificial Sequence
  artificial antibody sequence
GNSNRPS

SEQ ID NO: 74 - protein, 10 aa, Artificial Sequence
  artificial antibody sequence
SSYTQNSTRL

SEQ ID NO: 75 - protein, 110 aa, Artificial Sequence
  artificial antibody sequence
QSVLTQPPSASGTPGQRVTISCTGSSSNIGAGYDVHWYQQLPGTAPKLLIYGNSNRPSGVPDRFSGSKSGTSASLAISGLRSEDEADYYCSSYTQNSTRLFGGGTKLTVL

SEQ ID NO: 76 - protein, 5 aa, Artificial Sequence
  artificial antibody sequence
SYSMH

SEQ ID NO: 77 - protein, 17 aa, Artificial Sequence
  artificial antibody sequence
AISGSGGSTYYADSVKG

SEQ ID NO: 78 - protein, 12 aa, Artificial Sequence
  artificial antibody sequence
TPRRWGWSALDY

SEQ ID NO: 79 - protein, 121 aa, Artificial Sequence
  artificial antibody sequence
EVQLLESGGGLVQPGGSLRLSCAASGFTFSSYSMHWVRQGPGKGLEWVSAISGSGGSTYYADSVKGRFTISRDNSKNTLYLQMNSLRAEDTAVYYCARTPRRWGWSALDYWGQGTLVTVSS

SEQ ID NO: 80 - protein, 14 aa, Artificial Sequence
  artificial antibody sequence
TGSSSNIGAGYDVH

SEQ ID NO: 81 - protein, 7 aa, Artificial Sequence
  artificial antibody sequence
GNSNRPS

SEQ ID NO: 82 - protein, 11 aa, Artificial Sequence
  artificial antibody sequence
AAWDDSVSGWV

SEQ ID NO: 83 - protein, 111 aa, Artificial Sequence
  artificial antibody sequence
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSVSGWVFGGGTKLTVL

SEQ ID NO: 84 - protein, 5 aa, Artificial Sequence
  artificial antibody sequence
SYAMS

SEQ ID NO: 85 - protein, 17 aa, Artificial Sequence
  artificial antibody sequence
AISGSGGSTYYADSVKG

SEQ ID NO: 86 - protein, 13 aa, Artificial Sequence
  artificial antibody sequence
EVWGYSGYDYVDY

SEQ ID NO: 87 - protein, 122 aa, Artificial Sequence
  artificial antibody sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAREVWGYSGYDYVDYWGQGTLVTVSS

SEQ ID NO: 88 - protein, 216 aa, Artificial Sequence
  artificial antibody sequence
QSVLTQPPSASGTPGQRVTISCSGSSSNIGSNYVYWYQQLPGTAPKLLIYRNNQRPSGVPDRFSGSKSGTSASLAISGLRSEDEADYYCTAWDDSLSAVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 89 - protein, 448 aa, Artificial Sequence
  artificial antibody sequence
EVQLLESGGGLVQPGGSLRLSCAASGFTFSNYVMSWVRQAPGKGLEWVSGVSWNGSRTHYVDSVKRRFTISRDNSKNTLYLQMNSLRAEDTAVYYCARGLRYSSPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 216 aa, Artificial Sequence
  artificial antibody sequence
QSVLTQPPSASGTPGQRVTISCSGSSSNIGSNTVNWYQQLPGTAPKLLIYGNSNRPSGVPDRFSGSKSGTSASLAISGLRSEDEADYYCAVWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 91 - protein, 451 aa, Artificial Sequence
  artificial antibody sequence
EVQLLESGGGLVQPGGSLRLSCAASGFTFSRYGIHWVRQAPGKGLEWVAVISYDGTNKYYADSVKGRFTISRDNSKNTLYLQMNSLRAEDTAVYYCARARSRWASLGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 218 aa, Artificial Sequence
  artificial antibody sequence
QSVLTQPPSASGTPGQRVTISCSGSGSNIGNNAVNWYQQLPGTAPKLLIYGNSNRPSGVPDRFSGSKSGTSASLAISGLRSEDEADYYCQSYGTSLSGSRVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 93 - protein, 454 aa, Artificial Sequence
  artificial antibody sequence
EVQLLESGGGLVQPGGSLRLSCAASGFTFRKYWMHWVRQTPGKGLEWVSSVSASGGSIYYADSVRGRFTISRDNSKNTLYLQMNSLRAEDTAVYYCARGPFWSGYYRLDGLVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 215 aa, Artificial Sequence
  artificial antibody sequence
QSVLTQPPSASGTPGQRVTISCSGSSSNIGNNAVNWYQQLPGTAPKLLIYRDDRLPSGVPDRFSGSKSGTSASLAISGLRSEDEADYYCSSYTTSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 95 - protein, 448 aa, Artificial Sequence
  artificial antibody sequence
EVQLLESGGGLVQPGGSLRLSCAASGFTFSRYAMSWVRQAPGKGLEWVSGVSWNGSRTHYVGSVKRRFTISRDNSKNTLYLQMNSLRAEDTAVYYCARERLGKWYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 215 aa, Artificial Sequence
  artificial antibody sequence
QSVLTQPPSASGTPGQRVTISCSGSRSNIGSSVVSWYQQLPGTAPKLLIYGNNQRPSGVPDRFSGSKSGTSASLAISGLRSEDEADYYCTSYAGSNNLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - protein, 448 aa, Artificial Sequence
  artificial antibody sequence
EVQLLESGGGLVQPGGSLRLSCAASGFTFSSYSMNWVRQAPGKGLEWVSYISRSSGAIYYADSVKGRFTISRDNSKNTLYLQMNSLRAEDTAVYYCARERLGKWYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 216 aa, Artificial Sequence
  artificial antibody sequence
QSVLTQPPSASGTPGQRVTISCTGSSSNIGAGYDVHWYQQLPGTAPKLLIYGNSNRPSGVPDRFSGSKSGTSASLAISGLRSEDEADYYCSSYTQNSTRLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - protein, 450 aa, Artificial Sequence
  artificial antibody sequence
EVQLLESGGGLVQPGGSLRLSCAASGFTFSSYSMHWVRQGPGKGLEWVSAISGSGGSTYYADSVKGRFTISRDNSKNTLYLQMNSLRAEDTAVYYCARTPRRWGWSALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 217 aa, Artificial Sequence
  artificial antibody sequence
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSVSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 101 - protein, 451 aa, Artificial Sequence
  artificial antibody sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAREVWGYSGYDYVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2022/0128561 A1
(43) Pub. Date: Apr. 28, 2022

(54) METHODS TO DETERMINE WHETHER A SUBJECT IS SUITABLE OF BEING TREATED WITH AN AGONIST OF SOLUBLE GYANYLYL CYCLASE (SGC)

(71) Applicant: Bayer Aktiengesellschaft, Leverkusen (DE)

(72) Inventors: Rene Hoet, LG Boxmeer (NL); Peter Sandner, Wuppertal (DE); Jan Robert Krähling, Düsseldorf (DE); Agnes Benardeau, Düsseldorf (DE); Ana Lúcia Freitas de Mesquita Barbas, Carcavelos (PT); Ligia Nobre, Lousa (PT); Hugo Soares, Amadora (PT); Gabriela Silva, Carnaxide (PT)

(73) Assignee: Bayer Aktiengesellschaft, Leverkusen (DE)

(21) Appl. No.: 17/423,373

(22) PCT Filed: Jan. 16, 2020
(86) PCT No.: PCT/EP2020/051015

§ 371 Date: Jul. 15, 2021

(30) Foreign Application Priority Data
Jan. 17, 2019 (EP) 19152338.0

(51) Int. Cl. G01N 33/573; C07K 16/40; C12N 15/115

(52) U.S. Cl. CPC G01N 33/573; C07K 16/40; G01N 2333/988; C07K 2317/565; C07K 2317/56; C12N 15/115

(57) ABSTRACT

The present invention provides a method for determining whether a human or animal subject suffers from oxidative stress, is suitable of being treated with an antioxidant and/or free radical scavenger, and/or is suitable of being treated with an agonist of soluble Guanylyl Cyclase (sGC), in particular with an activator of sGC, said method comprising the steps of providing a tissue or liquid sample from said subject, and determining whether or not said sample is characterized by the presence, upregulation or overexpression of sGC comprising a heme free β1 subunit.

The present invention provides a method for determining whether a human or animal subject suffers from oxidative stress, is suitable of being treated with an antioxidant and/or free radical scavenger, and/or is suitable of being treated with an agonist of soluble Guanylyl Cyclase (sGC), in particular with an activator of soluble Guanylyl Cyclase (sGC).

### BACKGROUND OF THE INVENTION

The Nitric Oxide (NO), cyclic guanosine monophosphate (cGMP) pathway (NO/cGMP pathway) is of paramount importance for the regulation of cell, tissue and organ function and plays a major role in health and diseases. It is well established that the NO/cGMP pathway plays a critical role in diseases, including heart, kidney, lung, cardiovascular, cardiorenal and cardiopulmonary diseases, such as heart failure, chronic and acute kidney disease, and pulmonary hypertension. This is confirmed by genetic evidence, e.g. from genome wide association studies (GWAS) which showed strong correlation of genetic alterations in this pathway with a variety of diseases.

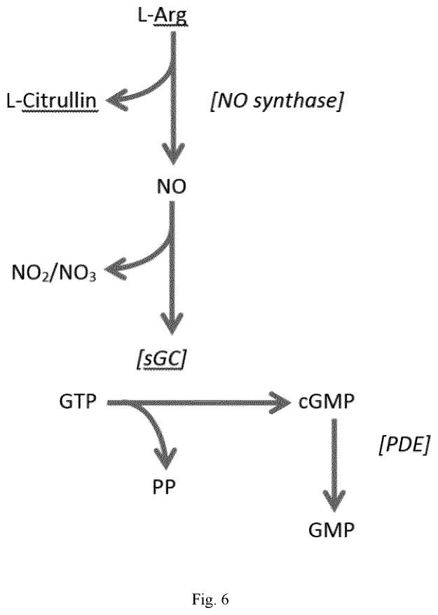

In short, the pathway is as follows (see also FIG. 6):
1. NO is formed from L-Arginine, e.g. due to endothelial shear stress, catalyzed by NO synthases
2. NO diffuses into the cell, and binds to the heme moiety of the β-subunit of the soluble Guanylyl Cyclase (sGC)
3. NO-binding to the sGC activates the enzyme, which then catalyzes the formation of cGMP out of GTP
4. cGMP acts as 2^{nd} messenger on multiple downstream targets, like cGMP regulated proteinkinases (PKGs, cGK-I/cGK-II), cGMP regulated ion channel and cGMP-regulated phosphodiesterases (PDEs) and further downstream targets which are phosphorylated and/or dephosphorylated
5. cGMP is hydrolyzed to inactive GMP by phosphodiesterases (PDEs) terminating NO/cGMP signaling

Since NO/cGMP plays a critical role in cell, tissue and body homeostasis, a decrease of cGMP levels can have unwanted or even pathophysiological consequences. Therapeutic approaches to address this condition encompass
administration of Nitrates or NO donors, e.g., in the treatment of angina pectoris. The respective agents release NO enzymatically or non-enzymatically, which binds to sGC and activate the latter, leading to an increased cGMP production. This approach has some shortcomings, like radical formation, development of tachyphylaxia, and kinetic limitations.
administration of PDE inhibitors, like Sildenafil, Vardenafil or Tadalafil. These agents have been used in the treatment of erectile dysfunction (ED), pulmonary arterial hypertension (PAH) and to treat signs and symptoms of benign prostatic hyperplasia (BPH). This approach has some shortcomings, too, like the demand of a sufficiently high NO production and high endogenous cGMP levels, which frequently are low in patients suffering from ED, PAH, or BPH.

To overcome the said limitations, attempts have been made to stimulate or activate the sGC directly with a suitable agent. This approach has the advantages that it is NO-independent, that there is no radical formation, and that it is not dependent on a sufficiently high cGMP level in the patient.

sGC is a heterodimer composed of one alpha and one heme containing β subunit. The β subunit consists of four domains: an N-terminal HNOX domain, a PAS-like domain, a coiled-coil domain, and a C-terminal catalytic domain. The HNOX domain of the β subunit contains a heme moiety with a Fe(II), which is the target of NO. Upon NO-binding, there is an increase in sGC activity, and cGMP is formed. sGC comprising a heme free β1 subunit is also called apo-sGC.

The HNOX (Heme Nitric oxide/OXygen binding) domain of the β subunit of sGC contains the prosthetic heme group and is part of a family of related sensor proteins found throughout a wide range of organisms. The HNOX domain uses the bound heme to sense gaseous ligands such as NO.

It is well accepted, that sGC stimulators act via direct stimulation of the sGC which does not require NO but requires the prosthetic heme-group. Therefore, this compound class of sGC stimulators is defined as NO-independent but heme-dependent sGC stimulators. The sGC stimulators bind to the alpha subunit of the non-oxidized and heme containing sGC (al/B1), also termed wild type sGC which leads to NO-independent formation and increase of intracellular cGMP (Stasch et al. 2001; Stasch & Hobbs 2009). In addition, sGC stimulators enhance the NO-effect on cGMP when NO is bound to the sGC. Therefore, sGC stimulators also exhibit synergistic effects with NO on cGMP production. The indazole derivative YC-1 was the first NO-independent but heme-dependent sGC stimulator described [Evgenov et al., 2006.]. Based on YC-1, further substances were discovered which are more potent than YC-1 and show no relevant inhibition of phosphodiesterases (PDE). This led to the identification of the pyrazolopyridine derivatives BAY 41-2272, BAY 41-8543 and BAY 63-2521 (Riociguat) [Evgenov et al., ibid.]. More recently other compound classes were discovered with differences in pharmacokinetics but also different organ distribution which might have impact on their treatment potential [Follmann et al. J. Med Chem 2017]. The exact binding site of the sGC stimulators at the wild type sGC is still being debated. If the heme group is removed from the sGC, the enzyme still has a detectable catalytic basal activity, i.e. cGMP is still being formed. The remaining catalytic basal activity of the heme free enzyme cannot be stimulated by any of the stimulators mentioned above and can also not be stimulated by NO [Evgenov et al., ibid.].

This observation is important since heme free and oxidized forms of the sGC (al/B1), also termed apo-sGC, are preferentially present at diseases which are linked to oxidative stress and other conditions The current understanding is that under oxidative stress conditions, the Fe²⁺ iron atom of the heme group in the β1 subunit is oxidized to Fe′ which destabilizes the binding of the heme group to the β1 subunit and renders the enzyme heme free. With the discovery of BAY 58-2667 (Cinaciguat), a new chemical matter has found which is able to activate heme free apo-sGC. Therefore BAY 58-2667 is the prototype of this class of sGC activators and this compound class is defined as NO-independent and heme-independent sGC activators. Common characteristics of these substances are that in combination with NO they only have an additive effect on enzyme activation, and that the activation of the oxidized or heme free enzyme is markedly higher than that of the heme containing enzyme [Evgenov et al., ibid.; J. P. Stasch et al., Br. J. Pharmacol. 136 (2002), 773; J. P. Stasch et al., J. Clin. Invest. 116 (2006), 2552]. Spectroscopic studies show that BAY 58-2667 displaces the oxidized heme group in the β1 subunit which, as a result of the weakening of the iron-histidine bond, is attached only weakly to the sGC. It has also been shown that the characteristic sGC heme binding motif Tyr-x-Ser-x-Arg is absolutely essential both for the interaction of the negatively charged propionic acids of the heme group and for the action of BAY 58-2667. Therefore, it is assumed that the binding site of BAY 58-2667 at the sGC is identical to the binding site of the heme group in the β1 subunit. [J. P. Stasch et al., J. Clin. Invest. 116 (2006), 2552]. More recently other classes of sGC activators have been discovered which are different in pharmacokinetics but also in organ distribution which might impact on their treatment potential.

It is another object of the present invention to provide tools and methods to identify patients that suffer from oxidative stress, and/or are suitable of being treated with an antioxidant and/or free radical scavenger

It is another object of the present invention to provide tools and methods to identify patients are suitable of being treated with an agonist of soluble Guanylyl Cyclase (sGC), in particular with an activator of sGC.

### SUMMARY OF THE INVENTION

These and further objects are met with methods and means according to the independent claims of the present invention. The dependent claims are related to specific embodiments.

### EMBODIMENTS OF THE INVENTION

Before the invention is described in detail, it is to be understood that this invention is not limited to the particular component parts or structural features of the devices or compositions described or process steps of the methods described as such devices and methods may vary. It is also to be understood that the terminology used herein is for purposes of describing particular embodiments only, and is not intended to be limiting. The mere fact that certain measures are recited in mutually different dependent claims does not indicate that a combination of these measures cannot be used to advantage. Any reference signs in the claims should not be construed as limiting the scope. It must be noted that, as used in the specification and the appended claims, the singular forms “a,” “an” and “the” include singular and/or plural referents unless the context clearly dictates otherwise. Further, in the claims, the word “comprising” does not exclude other elements or steps.

It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the length of this specification manageable. It is further to be understood that the content of the prior art documents referred to herein is incorporated by reference, e.g., for enablement purposes, namely when e.g. a method is discussed details of which are described in said prior art document. This approach serves to keep the length of this specification manageable.

According to one aspect of the invention, a method for determining whether a human or animal subject
suffers from oxidative stress
is suitable of being treated with an antioxidant and/or free radical scavenger, and/or
is suitable of being treated with an agonist of soluble Guanylyl Cyclase (sGC), in particular with an activator of sGC,
   is provided, said method comprising the steps of
a) providing a tissue or liquid sample from said subject, and
b) determining whether or not said sample is characterized by the presence, overexpression or upregulation of sGC comprising a heme free β1 subunit.

According to one aspect of the invention, a method for determining whether a human or animal subject
suffers from oxidative stress
is suitable of being treated with an antioxidant and/or free radical scavenger, and/or
is suitable of being treated with an activator of soluble Guanylyl Cyclase (sGC),
   is provided, said method comprising the steps of
a) providing a tissue or liquid sample from said subject, and
b) determining whether or not said sample is characterized by the presence, overexpression or upregulation of sGC comprising a heme free β1 subunit.

This includes a method for determining whether a human or animal subject suffers from oxidative stress/disturbances from normal redox state of cells/imbalance between reactive oxygen species and capacity of the body to detoxify them, said method comprising the steps of
providing a tissue or liquid sample from said subject, and
determining whether or not said sample is characterized by the presence, overexpression or upregulation of an sGC comprising a heme free β1 subunit.

This further includes a method for determining whether a human or animal subject is suitable of being treated with an antioxidant and/or free radical scavenger, said method comprising the steps of
providing a tissue or liquid sample from said subject, and
determining whether or not said sample is characterized by the presence, overexpression or upregulation of an sGC comprising a heme free β1 subunit.

This further includes a method for determining whether a human or animal subject is suitable of being treated with an activator of soluble Guanylyl Cyclase (sGC), said method comprising the steps of
providing a tissue or liquid sample from said subject, and
determining whether or not said sample is characterized by the presence, overexpression or upregulation of an sGC comprising a heme free β1 subunit.

As used herein, the term “presence of sGC comprising a heme free β1 subunit” means that in said sample, such sGC comprising a heme free β1 subunit can be determined by histochemical, immunologic or molecular methods.

As used herein, the term “overexpression of sGC comprising a heme free β1 subunit” refers to the level of sGC comprising a heme free β1 subunit expressed in cells of a given tissue being elevated in comparison to the levels thereof as measured in normal cells (free from disease) of the same type of tissue, under analogous conditions by at least 5%, preferably by at least 10%, more preferably by at least 15%, even more preferably by at least 20%, even more preferably by at least 25%, even more preferably by at least 30% or by at least 40% or by at least 50%. Said expression level may be determined by a number of techniques known in the art including, but not limited to, quantitative RT-PCR, western blotting, immunohistochemistry, and suitable derivatives of the above.

As used herein, the term “upregulation of sGC comprising a heme free β1 subunit” refers to the gene regulation of the expression of sGC comprising a heme free β1 subunit in cells of a given tissue being elevated in comparison to the levels thereof as measured in normal cells (free from disease) of the same type of tissue, under analogous conditions by at least 5%, preferably by at least 10%, more preferably by at least 15%, even more preferably by at least 20%, even more preferably by at least 25%, even more preferably by at least 30% or by at least 40% or by at least 50%.

As used herein, the term “oxidative stress is defined as a disturbance in the balance between the production of reactive oxygen species (free radicals, ROS) and antioxidant defenses. ROS comprise but are not limited to superoxide anion •O⁻, to hydrogen peroxide H₂O₂, to hydroxyl radicals •OH, to organic hydroperoxide ROOH, to alkoxy and peroxy radicals RO• and ROO•, to peroxynitrite ONOO⁻.

As used herein, the term “antioxidans” relates to a molecule that is capable to inhibit oxidation of another entity. Oxidation is a chemical reaction that can produce free radicals, thereby leading to chain reactions that may damage the cells of organisms. Antioxidants such as thiols or ascorbic acid (vitamin C) terminate these chain reactions. Antioxidants can be subgrouped into primary antioxidants and secondary antioxidants. Biological antioxidants include well-defined enzymes, such as superoxide dismutase, catalase, selenium glutathione peroxidase, and phospholipid hydroperoxide glutathione peroxidase. Nonenzymatic biological antioxidants include tocopherols and tocotrienols, carotenoids, quinones, bilirubin, ascorbic acid, uric acid, and metal-binding proteins. Various antioxidants, being both lipid and water soluble, are found in all parts of cells and tissues, although each specific antioxidant often shows a characteristic distribution pattern. The so-called ovothiols, which are mercaptohistidine derivatives, also decompose peroxides nonenzymatically.

As used herein, the term “free radical scavenger” relates to a subgroup of antioxidants, which is capable of binding and detoxifying free radicals. Examples include buthionine sulphoximine, vitamin C, indomethacin, ibuprofen, N-acetyl cysteine, or aspirin.

According to one embodiment of the invention, said activator of soluble Guanylyl Cyclase (sGC) is a molecule that activates the oxidized, heme free sGC heterodimer (α1/β1 or α2/β1), to catalyze the formation of cGMP.

As used herein, an “activator”, “activator of soluble Guanylyl Cyclase (sGC)”, “sGC activator”, or “heme-independent sGC activator” is an active compound that interacts with an oxidized or heme-free form of the sGC, to activate an oxidized or heme-free form of the sGC to catalyze the formation of cGMP. It is to be understood as a compound increasing the measured production of cGMP by at least 5% as compared to a control, e.g., a non-treated control, preferably by at least 10%, more preferably by at least 15%, even more preferably by at least 20%, even more preferably by at least 25%, even more preferably by at least 30% or by at least 40% or by at least 50%. Suitable controls are evident for the skilled person when considering the teaching of the present disclosure. Suitable assays to determine said activation are readily available to the skilled person from the pertinent literature. In one embodiment of the invention, the assay “Activation of recombinant soluble guanylate cyclase (sGC) in vitro” described below is being used to determine said activation. This test is suitable to distinguish between the heme-dependent sGC Stimulators and the heme-independent sGC Activators.

Preferably, the soluble Guanylyl Cyclase is human soluble Guanylyl Cyclase.

According to one embodiment of the invention, said activator of soluble Guanylyl Cyclase (sGC) is at least one selected from the list comprising
4-({(4-carboxybutyl)[2-(2-{[4-(2-phenylethyl)benzyl]oxy}phenyl)ethyl]amino}methyl)benzoic acid
5-chloro-2-(5-chlorothiophene-2-sulfonylamino-N-(4-(morpholine-4-sulfonyl)phenyl)benzamide as sodium salt
2-(4-chlorophenylsulfonylamino)-4,5-dimethoxy-N-(4-(thiomorpholine-4-sulfonyl)phenyl)benzamide
1-{6-[5-chloro-2-({4-trans-4-}trifluoromethyl)cyclohexyl]benzyl}oxy)phenyl]pyridin-2-yl}-5-(trifluoromethyl)-1H-pyrazole-4-carboxylic acid
1-[6-(2-(2-methyl-4-(4-trifluoromethoxyphenyl)benzyloxy)phenyl)pyridin-2-yl]-5-trifluoromethylpyrazole-4-carboxylic acid
1[6-(3,4-dichlorophenyl)-2-pyridinyl-5-(trifluoromethyl)-1H-pyrazole-4-carboxylic acid
1-({2-[3-chloro-5-(trifluoromethyl)phenyl]-5-methyl-1,3-thiazol-4-yl}methyl)-1H-pyrazole-4-carboxylic acid
4-({2-[3-(trifluoromethyl)phenyl]-1,3-thiazol-4-yl}methyl)benzoic acid
1-({2-[2-fluoro-3-(trifluoromethyl)phenyl]-5-methyl-1,3-thiazol-4-yl}methyl)-1H-pyrazole-4-carboxylic acid
3-(4-chloro-3-{[(2S,3R)-2-(4-chlorophenyl)-4,4,4-trifluoro-3-methylbutanoyl]amino}phenyl)-3-cyclopropylpropanoic acid
5-{[2-(4-carboxyphenyl)ethyl][2-(2-{[3-chloro-4′-(trifluoromethyl)biphenyl-4-yl]methoxy}phenyl)ethyl]amino}-5,6,7,8-tetrahydroquinoline-2-carboxylic acid formula
5-{(4-carboxybutyl)[2-(2-{[3-chloro-4′-(trifluoromethyl)biphenyl-4-yl]methoxy}phenyl)ethyl]amino}-5,6,7,8-tetrahydroquinoline-2-carboxylic acid of the formula
(1R,5S)-3-[4-(5-methyl-2-{[2-methyl-4-(piperidin-1-ylcarbonyl)benzyl]oxy}phenyl)-1,3-thiazol-2-yl]-3-azabicyclo[3.2.1]octane-8-carboxylic acid
1-[6-(5-methyl-2-{[2-(tetrahydro-2H-pyran-4-yl)-1,2,3,4-tetrahydroisoquinolin-6-yl]methoxy}phenyl)pyridin-2-yl]-5-(trifluoromethyl)-1H-pyrazole-4-carboxylic acid
4-[[(4-Carboxybutyl)[2-[2-[[4-(2-phenylethyl)phenyl]methoxy]phenyl]ethyl]amino]methyl]benzoic acid
BAY 60-2770 4-({(4-carboxybutyl) [2-(5-fluoro-2-{[40-(trifluoromethyl) biphenyl-4-yl]methoxy}phenyl)ethyl]amino}methyl)benzoic acid)

Further sGC activators in the context of the invention are disclosed in one of the following publications: WO2013/157528, WO2015/056663, WO2009/123316, WO2016/001875, WO2016/001876, WO2016/001878, WO2000/02851, WO2012/122340, WO2013/025425, WO2014/039434, WO2016/014463, WO2009/068652, WO2009/071504, WO2010/015652, WO2010/015653, WO2015/033307, WO2016/042536, WO2009/032249, WO2010/099054, WO2012/058132, US2010/0216764, WO01/19776, WO01/19780, WO01/19778, WO02/070459, WO02/070460, WO02/070510, WO02/070462, WO2007/045366, WO2007/045369, WO2007/045433, WO2007/045370, WO2007/045367, WO2014/012935, WO2014/012934, WO2011/141409, WO2008/119457, WO2008/119458, WO2009/127338, WO2010/102717, WO2011/051165, WO2012/076466, WO2012/139888, WO2013/157528, WO2013/174736, WO2014/012934, WO2015/056663, WO2017103888, WO2017112617, WO2016042536, WO2016081668, WO2016191335, WO2016191334, WO2016001875, WO2016001876, WO2016001878, WO2016014463, WO2016044447, WO2016044445, WO2016044446, WO2015056663, WO2015033307, WO2015187470, WO2015088885, WO2015088886, WO2015089182, WO2014084312, WO2014039434, WO2014144100, WO2014047111, WO2014047325, WO2013025425, WO2013101830, WO2012165399, WO2012058132, WO2012122340, WO2012003405, WO2012064559, WO2011149921, WO2011119518, WO2011115804, WO2011056511, CN101670106, TW201028152, WO2010015653, WO2010015652, WO2010099054, WO2010065275, WO2009123316, WO2009068652, WO2009071504, WO2009032249, US2009209556.

According to one embodiment of the invention, in the step for determining whether or not said sample is characterized by the presence, upregulation or overexpression of sGC comprising a heme free β1 subunit, a binding molecule is used which selectively binds to sGC comprising a heme free β1 subunit.

As used herein, the term “selectively binds to sGC comprising a heme free β1 subunit” means that such binding molecule has significantly higher binding affinity and/or selectivity to (i) sGC comprising a heme free β1 subunit than to (ii) wildtype sGC, comprising a native, heme-comprising β subunit.

As used herein, the term “binding affinity” refers to the affinity of a binding molecule according to the invention, to its target, sGC comprising a heme free β1 subunit, and is expressed numerically using “K_{D}” values. In general, a higher K_{D} value corresponds to a weaker binding. In some embodiments, the “K_{D}” is measured by a radiolabeled antigen binding assay (MA) or surface plasmon resonance (SPR) assays, using, e.g., a BIAcore™-2000 or a BIAcore™-3000 In certain embodiments, an “on-rate” or “rate of association” or “association rate” or “kₒₙ” and an “off-rate” or “rate of dissociation” or “dissociation rate” or “k_{off}” are also determined with the surface plasmon resonance (SPR) technique. In additional embodiments, the “K_{D}”, “kₒₙ”, and “k_{off}” are measured using the Octet® Systems (Pall Life Sciences).

As used herein, the term “selectivity” describes the characteristic of a binding molecule according to the invention, to bind its target, sGC comprising a heme free β1 subunit, with a K_{D} about 1000-, 500-, 200-, 100-, 50-, or about 10-fold lower than it binds other proteins, including a native, heme-comprising β subunit, as e.g. measured by surface plasmon resonance (SPR).

As used herein, the terms “higher binding affinity” and “higher selectivity” of the binding molecule according to the invention imply that the respective parameter of the binding molecule according to the invention is at least 5% higher with regard to sGC comprising a heme free β1 subunit than with regard to a native, heme-comprising β subunit, preferably at least 10%, more preferably at least 15%, even more preferably at least 20%, even more preferably at least 25%, even more preferably at least 30% or at least 40% or at least 50%.

According to one embodiment of the invention, said binding molecule is an antibody, or fragment or derivative thereof retaining target binding capacity, an antibody mimetic, or an aptamer.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

The term “antibody”, as used herein, is intended to refer to immunoglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs arranged from amino-terminus to carboxy-terminus e.g. in the following order: FR1, CDR1, FR2, CDR2, FR3, CDR3, FR4.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H3) in the heavy chain variable domain; (Kabat et al., Sequences of Proteins of Immulological Interest, 5th Ed. Public Health Service, National Institutes of Health, Bethesda, Md. (1991)) and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain (Chothia and Lesk; J Mol Biol 196: 901-917 (1987)). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA1, and IgA2. A preferred class of immunoglobulins for use in the present invention is IgG.

The heavy-chain constant domains that correspond to the different classes of antibodies are called [alpha], [delta], [epsilon], [gamma], and [mu], respectively. The subunit structures and three-dimensional configurations of different classes of immunoglobulins are well known. As used herein antibodies are conventionally known antibodies and functional fragments thereof.

A “functional fragment” or “antigen-binding antibody fragment” or “fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320).

Examples are
a CDR (complementarity determining region),
a hypervariable region,
a variable domain (Fv),
an IgG heavy chain (consisting of VH, CH1, hinge, CH2 and CH3 regions),
an IgG light chain (consisting of VL and CL regions), and/or
a Fab and/or F(ab)₂.

“Functional fragments”, “antigen-binding antibody fragments”, or “antibody fragments” of the invention include but are not limited to Fab, Fab′, Fab′-SH, F(ab′)₂, and Fv fragments; diabodies; single domain antibodies (DAbs), linear antibodies; single-chain antibody molecules (scFv); and multispecific, such as bi- and tri-specific, antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “multi-specific” or “multi-functional” antibody is understood to have each of its binding sites identical. The F(ab′)₂ or Fab may be engineered to minimize or completely remove the intermolecular disulfide interactions that occur between the CH1 and CL domains.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Variants of the antibodies or antigen-binding antibody fragments contemplated in the invention are molecules in which the binding activity of the antibody or antigen-binding antibody fragment is maintained.

“Binding proteins” contemplated in the invention are for example antibody mimetics, such as Affibodies, Adnectins, Anticalins, DARPins, Avimers, Nanobodies (reviewed by Gebauer M. et al., Curr. Opinion in Chem. Biol. 2009; 13:245-255; Nuttall S. D. et al., Curr. Opinion in Pharmacology 2008; 8:608-617).

A “human” antibody or antigen-binding fragment thereof is hereby defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source). Examples of human antibodies include antibodies as described in Söderlind et al., Nature Biotech. 2000, 18:853-856.

A “humanized antibody” or humanized antigen-binding fragment thereof is defined herein as one that is (i) derived from a non-human source (e.g., a transgenic mouse which bears a heterologous immune system), which antibody is based on a human germline sequence; (ii) where amino acids of the framework regions of a non-human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is defined herein as one, wherein the variable domains are derived from a non-human origin and some or all constant domains are derived from a human origin.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

An “isolated antibody” is one that has been identified and separated from a component of the cell that expressed it. Contaminant components of the cell are materials that would interfere with diagnostic or therapeutic uses of the antibody, and may include enzymes, hormones, and other proteinaceous or nonproteinaceous solutes.

As used herein, an antibody “binds specifically to”, is “specific to/for” or “specifically recognizes” an antigen of interest, e.g. a tumor-associated polypeptide antigen target, is one that binds the antigen with sufficient affinity such that the antibody is useful as a therapeutic agent in targeting a cell or tissue expressing the antigen, and does not significantly cross-react with other proteins or does not significantly cross-react with proteins other than orthologs and variants (e.g. mutant forms, splice variants, or proteolytically truncated forms) of the aforementioned antigen target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to surface plasmon resonance (SPR), Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

As used herein, the term “epitope” includes any protein determinant capable of specific binding to an immunoglobulin or T-cell receptor. Epitopic determinants usually consist of chemically active surface groupings of molecules such as amino acids or sugar side chains, or combinations thereof and usually have specific three dimensional structural characteristics, as well as specific charge characteristics.

An “antibody that binds to the same epitope” as a reference antibody or “an antibody which competes for binding” to a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

“Sequence homology” indicates the percentage of amino acids that either is identical or that represent conservative amino acid substitutions.

The term “maturated antibodies” or “maturated antigen-binding fragments” such as maturated Fab variants includes derivatives of an antibody or antibody fragment exhibiting stronger binding—i. e. binding with increased affinity—to a given antigen such as the extracellular domain of a target protein. Maturation is the process of identifying a small number of mutations e.g. within the six CDRs of an antibody or antibody fragment leading to this affinity increase. The maturation process is the combination of molecular biology methods for introduction of mutations into the antibody and screening for identifying the improved binders.

The term “pharmaceutical formulation”/“pharmaceutical composition” refers to a preparation which is in such form as to permit the biological activity of an active ingredient contained therein to be effective, and which contains no additional components which are unacceptably toxic to a subject to which the formulation would be administered.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell”, “host cell line”, and “host cell culture” are used interchangeably and refer to cells into which exogenous nucleic acid has been introduced, including the progeny of such cells. Host cells include “transformants”, “transformed cells”, “transfectants”, “transfected cells”, and “transduced cells”, which include the primary transformed/transfected/transduced cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

Antibodies differ in sequence, not only within their complementarity determining regions (CDRs), but also in the framework (FR). These sequence differences are encoded in the different V-genes. The human antibody germline repertoire has been completely sequenced. There are about 50 functional VH germline genes which can be grouped into six subfamilies according to sequence homology VH1, VH2, VH3, VH4, VH5 and VH6 (Tomlinson et al., 1992, J. Mol. Biol. 227, 776-798; Matsuda & Honjo, 1996, Advan. Immunol. 62, 1-29). About 40 functional VL kappa genes comprising seven subfamilies are known (Cox et al., 1994, Eur. J. Immunol. 24, 827-836; Barbie & Lefranc, 1998, Exp. Clin. Immunogenet. 15, 171-183): Vkappa1, Vkappa2, Vkappa3, Vkappa4, Vkappa5, Vkappa6 and Vkappa7. Disclosed herein are heavy chains of antibodies of this invention that belong to the human VH2 subfamily and the light chains of antibodies of this invention that belong to the human Vkappa1 subfamily, respectively. It is known that framework sequences of antibodies belonging to the same subfamily are closely related, e.g. antibodies comprising a human VH3 subfamily member all share comparable stability (Honegger et al., 2009, Protein Eng Des Sel. 22(3):121-134). It is well known in the art that CDRs from antibodies can be grafted on different frameworks while maintaining special features of the corresponding origin antibody. CDRs have been successfully grafted on frameworks belonging to a different species as well as on frameworks of the same species belonging to a different subfamily. In a further embodiment the antibody or antigen-binding fragment of the invention comprises at least one CDR sequence of antibody of the invention as depicted in Table 1 and a human variable chain framework sequence.

In a preferred embodiment the antibody or antigen-binding fragment of the invention comprises a variable light chain or light chain antigen-binding region comprising the L-CDR1, L-CDR2 and L-CDR3 sequence of the variable light chain and a variable heavy chain or heavy chain antigen-binding region comprising the H-CDR1, H-CDR2 and H-CDR3 sequence of the variable heavy chain antibody of the invention as depicted in Table 1 and a human variable light and human variable heavy chain framework sequence.

An antibody of the invention may be an IgG (e.g. IgG1 IgG2, IgG3, IgG4) or IgA, IgD, IgE, IgM, while an antibody fragment may be a Fab, Fab′, F(ab′)₂, Fab′-SH or scFv, for example. An inventive antibody fragment, accordingly, may be, or may contain, an antigen-binding region that behaves in one or more ways as described herein.

In a preferred embodiment the antibodies or antigen-binding antibody fragments of the invention are monoclonal.

In some embodiments antibodies of the invention or antigen-binding fragments thereof or nucleic acids encoding the same are isolated. An isolated biological component (such as a nucleic acid molecule or protein such as an antibody) is one that has been substantially separated or purified away from other biological components in the cell of the organism in which the component naturally occurs, e.g., other chromosomal and extra-chromosomal DNA and RNA, proteins and organelles. The term also embraces nucleic acids and proteins prepared by recombinant expression in a host cell as well as chemically synthesized nucleic acids.

Aptamers are oligonucleotides that have specific binding properties for a pre-determined target. They are obtained from a randomly synthesized library containing up to 10¹⁵ different sequences through a combinatorial process named SELEX (“Systematic Evolution of Ligands by EXponential enrichment”). Aptamer properties are dictated by their 3D shape, resulting from intramolecular folding, driven by their primary sequence. An aptamer3D structure is exquisitely adapted to the recognition of its cognate target through hydrogen bonding, electrostatic and stacking interactions. Aptamers generally display high affinity (K_{d} about micromolar (μM) for small molecules and picomolar (pM) for proteins). An overview on the technical repertoire to generate target specific aptamers is given, e.g., in Blind and Blank 2015, which is incorporated herein by reference. Aptamers can also be delivered into the intracellular space, as disclosed in Thiel and Giangrande (2010), incorporated herein by reference.

### Antibody Generation

An antibody of the invention may be derived from a recombinant antibody library that is based on amino acid sequences that have been isolated from the antibodies of a large number of healthy volunteers e.g. using the n-CoDeR® technology the fully human CDRs are recombined into new antibody molecules (Carlson & Söderlind, Expert Rev Mol Diagn. 2001 May; 1(1):102-8). Or alternatively for example antibody libraries as the fully human antibody phage display library described in Hoet R M et al., Nat Biotechnol 2005; 23(3):344-8) can be used to isolate (Apo-sGC)-specific antibodies. Antibodies or antibody fragments isolated from human antibody libraries are considered human antibodies or human antibody fragments herein.

Human antibodies may be further prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. For example immunization of genetically engineered mice inter alia immunization of hMAb mice (e.g. VelocImmune Mouse® or XENOMOUSE®) may be performed.

Further antibodies may be generated using the hybridoma technology (for example see Köhler and Milstein Nature. 1975 Aug. 7; 256(5517):495-7), resulting in for example murine, rat, or rabbit antibodies which can be converted into chimeric or humanized antibodies. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall' Acqua et al., Methods 36:43-60 (2005) (describing “FR shuffling”); and Osboum et al., Methods 36:61-68 (2005) and Klimka et al., Br. J. Cancer, 83:252-260 (2000) (describing the “guided selection” approach to FR shuffling).

Examples are provided for the generation of antibodies using a recombinant antibody library and immunization of mice combined with subsequent humanization.

### Peptide Variants

Antibodies or antigen-binding fragments of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies disclosed herein, while appreciating these variants having the ability to bind to apo-sGC fall within the scope of the present invention.

A variant can include, for example, an antibody that has at least one altered complementary determining region (CDR) (hyper-variable) and/or framework (FR) (variable) domain/position, vis-à-vis a peptide sequence disclosed herein.

By altering one or more amino acid residues in a CDR or FR region, the skilled worker routinely can generate mutated or diversified antibody sequences, which can be screened against the antigen, for new or improved properties, for example.

A further preferred embodiment of the invention is an antibody or antigen-binding fragment in which the VH and VL sequences are selected as shown in Table 2. The skilled worker can use the data in Table 2 to design peptide variants that are within the scope of the present invention. It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

Alternatively, the skilled worker could make the same analysis by comparing the amino acid sequences disclosed herein to known sequences of the same class of such antibodies, using, for example, the procedure described by Knappik A., et al., JMB 2000, 296:57-86.

Furthermore, variants may be obtained by using one antibody as starting point for further optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH. Diversification can be done e.g. by synthesizing a collection of DNA molecules using trinucleotide mutagenesis (TRIM) technology (Virnekäs B. et al., Nucl. Acids Res. 1994, 22: 5600.). Antibodies or antigen-binding fragments thereof include molecules with modifications/variations including but not limited to e.g. modifications leading to altered half-life (e.g. modification of the Fc part or attachment of further molecules such as PEG), altered binding affinity or altered ADCC or CDC activity.

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

### Glycosylation Variants

Where the antibody comprises an Fc region, the carbohydrate attached thereto may be altered. Native antibodies produced by mammalian cells typically comprise a branched, biantennary oligosaccharide that is generally attached by an N-linkage to Asn297 using Kabat EU numbering of the CH2 domain of the Fc region; see, e.g., Wright et al. Trends Biotechnol. 15: 26-32 (1997).

In certain embodiments, an antibody provided herein is altered to increase or decrease the extent to which the antibody is glycosylated. Addition or deletion of glycosylation sites to an antibody may be conveniently accomplished by altering the expression system (e.g. host cell) and/or by altering the amino acid sequence such that one or more glycosylation sites is created or removed.

In one embodiment of this invention, aglycosyl antibodies having decreased effector function or antibody derivatives are prepared by expression in a prokaryotic host. Suitable prokaryotic hosts for include but are not limited to E. coli, Bacillus subtilis, Salmonella typhimurium and various species within the genera Pseudomonas, Streptomyces, and Staphylococcus.

In one embodiment, antibody variants are provided having decreased effector function, which are characterized by a modification at the conserved N-linked site in the CH2 domains of the Fc portion of said antibody. In one embodiment of present invention, the modification comprises a mutation at the heavy chain glycosylation site to prevent glycosylation at the site. Thus, in one preferred embodiment of this invention, the aglycosyl antibodies or antibody derivatives are prepared by mutation of the heavy chain glycosylation site,—i.e., mutation of N297 using Kabat EU numbering and expressed in an appropriate host cell.

In another embodiment of the present invention, aglycosyl antibodies or antibody derivatives have decreased effector function, wherein the modification at the conserved N-linked site in the CH2 domains of the Fc portion of said antibody or antibody derivative comprises the removal of the CH2 domain glycans, —i.e., deglycosylation. These aglycosyl antibodies may be generated by conventional methods and then deglycosylated enzymatically. Methods for enzymatic deglycosylation of antibodies are well known in the art (e.g. Winkelhake & Nicolson (1976), J Biol Chem. 251(4):1074-80).

In another embodiment of this invention, deglycosylation may be achieved using the glycosylation inhibitor tunicamycin (Nose & Wigzell (1983), Proc Natl Acad Sci USA, 80(21):6632-6). That is, the modification is the prevention of glycosylation at the conserved N-linked site in the CH2 domains of the Fc portion of said antibody.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function.

Examples of publications related to “defucosylated” or “fucose-deficient” antibody variants include: Okazaki et al. J Mol. Biol. 336: 1239-1249 (2004); Yamane-Ohnuki et al. Biotech. Bioeng. 87: 614 (2004).

Examples of cell lines capable of producing defucosylated antibodies include Lec13 CHO cells deficient in protein fucosylation (Ripka et al. Arch. Biochem. Biophys. 249:533-545 (1986); and WO 2004/056312), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006)).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546.

Antibody variants with at least one galactose residue in the oligosaccharide attached to the Fc region are also provided. Such antibody variants may have improved CDC function. Such antibody variants are described, e.g., in WO1997/30087; WO1998/58964; and WO1999/22764.

### Fc Region Variants

In certain embodiments, one or more amino acid modifications (e.g. a substitution) may be introduced into the Fc region of an antibody (e.g., a human IgG1, IgG2, IgG3 or IgG4 Fc region) provided herein, thereby generating an Fc region variant.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability.

In some embodiments, alterations are made in the Fc region that result in altered (i.e., either improved or diminished) C1q binding and/or Complement Dependent Cytotoxicity (CDC).

In certain embodiments, the invention contemplates an antibody variant that possesses an increased or decreased half-live. Antibodies with increased half-lives and improved binding to the neonatal Fc receptor (FcRn), which is responsible for the transfer of maternal IgGs to the fetus (Guyer et al., J Immunol. 117:587 (1976) and Kim et al., J Immunol. 24:249 (1994)), are described in US2005/0014934 (Hinton et al.). Those antibodies comprise an Fc region with one or more substitutions therein which improve binding of the Fc region to FcRn.

### DNA Molecules of the Invention

The present invention also relates to the DNA molecules that encode an antibody of the invention or antigen-binding fragment thereof. These sequences are optimized in certain cases for mammalian expression. DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent DNA Variants

Yet another class of DNA variants within the scope of the invention may be described with reference to the product they encode. These functionally equivalent polynucleotides are characterized by the fact that they encode the same peptide sequences due to the degeneracy of the genetic code.

It is recognized that variants of DNA molecules provided herein can be constructed in several different ways. For example, they may be constructed as completely synthetic DNAs. Methods of efficiently synthesizing oligonucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In atypical method, a target DNA is cloned into a single-stranded DNA bacteriophage vehicle. Single-stranded DNA is isolated and hybridized with an oligonucleotide containing the desired nucleotide alteration(s). The complementary strand is synthesized, and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

The present invention further provides recombinant DNA constructs comprising one or more of the nucleotide sequences of the present invention. The recombinant constructs of the present invention can be used in connection with a vector, such as a plasmid, phagemid, phage or viral vector, into which a DNA molecule encoding an antibody of the invention or antigen-binding fragment thereof or variant thereof is inserted.

An antibody, antigen binding portion, or variant thereof provided herein can be prepared by recombinant expression of nucleic acid sequences encoding light and heavy chains or portions thereof in a host cell. To express an antibody, antigen binding portion, or variant thereof recombinantly a host cell can be transfected with one or more recombinant expression vectors carrying DNA fragments encoding the light and/or heavy chains or portions thereof such that the light and heavy chains are expressed in the host cell. Standard recombinant DNA methodologies are used to prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding fragments thereof or variants thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic hosts cells are yeasts, insects and insect cells, plants and plant cells, transgenic animals, or mammalian cells. In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

Therefore, an embodiment of the present invention are also host cells comprising the vector or a nucleic acid molecule, whereby the host cell can be a higher eukaryotic host cell, such as a mammalian cell, a lower eukaryotic host cell, such as a yeast cell, and may be a prokaryotic cell, such as a bacterial cell.

Another embodiment of the present invention is a method of using the host cell to produce an antibody and antigen binding fragments, comprising culturing the host cell under suitable conditions and recovering said antibody.

Therefore another embodiment of the present invention is the production of the antibodies according to this invention with the host cells of the present invention and purification of these antibodies to at least 95% homogeneity by weight.

### Bacterial Expression

Useful expression vectors for bacterial use are constructed by inserting a DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include but are not limited to E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and a bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore, an embodiment of the present invention is an expression vector comprising a nucleic acid sequence encoding for the novel antibodies of the present invention.

Antibodies of the present invention or antigen-binding fragments thereof or variants thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Expression of the antibodies may be constitutive or regulated (e.g. inducible by addition or removal of small molecule inductors such as Tetracyclin in conjunction with Tet system). For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and 5,179,017). Suitable selectable markers include genes that confer resistance to drugs such as G418, puromycin, hygromycin, blasticidin, zeocin/bleomycin or methotrexate or selectable marker that exploit auxotrophies such as Glutamine Synthetase (Bebbington et al., Biotechnology (N Y). 1992 February; 10(2):169-75), on a host cell into which the vector has been introduced. For example, the dihydrofolate reductase (DHFR) gene confers resistance to methotrexate, neo gene confers resistance to G418, the bsd gene from Aspergillus terreus confers resistance to blasticidin, puromycin N-acetyl-transferase confers resistance to puromycin, the Sh ble gene product confers resitance to zeocin, and resistance to hygromycin is conferred by the E. coli hygromycin resistance gene (hyg or hph). Selectable markers like DHFR or Glutamine Synthetase are also useful for amplification techniques in conjunction with MTX and MSX.

Transfection of the expression vector into a host cell can be carried out using standard techniques such as electroporation, nucleofection, calcium-phosphate precipitation, lipofection, polycation-based transfection such as polyethlylenimine (PEI)-based transfection and DEAE-dextran transfection.

Suitable mammalian host cells for expressing the antibodies, antigen binding fragments thereof or variants thereof provided herein include Chinese Hamster Ovary (CHO cells) such as CHO-K1, CHO-S, CHO-K1SV [including dhfr-CHO cells, described in Urlaub and Chasin, (1980) Proc. Natl. Acad. Sci. USA 77:4216-4220 and Urlaub et al., Cell. 1983 June; 33(2):405-12, used with a DHFR selectable marker, e.g., as described in R. J. Kaufman and P. A. Sharp (1982) Mol. Biol. 159:601-621; and other knockout cells exemplified in Fan et al., Biotechnol Bioeng. 2012 April; 109(4):1007-15], NS0 myeloma cells, COS cells, HEK293 cells, HKB11 cells, BHK21 cells, CAP cells, EB66 cells, and SP2 cells.

Expression might also be transient or semi-stable in expression systems such as HEK293, HEK293T, HEK293-EBNA, HEK293E, HEK293-6E, HEK293-Freestyle, HKB11, Expi293F, 293EBNALT75, CHO Freestyle, CHO-S, CHO-K1, CHO-K1SV, CHOEBNALT85, CHOS-XE, CHO-3E7 or CAP-T cells (for instance Durocher et al., Nucleic Acids Res. 2002 Jan. 15; 30(2):E9).

In some embodiments, the expression vector is designed such that the expressed protein is secreted into the culture medium in which the host cells are grown. The antibodies, antigen binding fragments thereof or variants thereof can be recovered from the culture medium using standard protein purification methods.

### Expression in Insect Cells

Expression of heterologous proteins in insect host cell includes the use of DNA vector-based expression such as recombinant plasmids or the use of viral-based expression systems such as the baculovirus expression system (BEVS). The transient expression of target proteins using insect virus-based vectors use regulatory sequences and derivatives from virus such as Autographa californica multicapsid nucleopolyhedrovirus (AcMNPV), Bombyx mori nucleopolyhedrovirus (BmNPV) and Orgyia pseudotsugata multicapsid nucleopolyhedrosis virus (OpMNPV). The preferred regulatory sequences for insect host cell expression include the use of BmNPV IE-1 transactivator, the BmNPV HR3 enhancer and the Bm cytoplasmic actin promoter (Farrell, Lu et al. 1998), the promoter region from Drosophila actin 5c gene (ac5) (Chung, Yang-Tsung et al. 1990), the OpIE2 promoter from OpMNPV, the polyhedrin (polh) and the IE1 promoters from AcMNPV, and the enhancer elements hr 1 to hr5 from AcMNPV (Ren, Linzhu et al. 2011).

Expression of antibodies or antigens may be constitutive or regulated (e.g. inducible by addition or removal of small molecule inductors such as Tetracyclin in conjunction with a wild-type or modified tetracycline-responsive expression system (TRES) for use in insect cells (Wu, Tzong-Yuan et al. 2000) or the addition of copper sulfate or cadmium chloride in conjunction with Drosophila metallothionein gene promoter (Bunch, Thomas et al. 1988)).

The recombinant expression vectors can also include origins of replication and selectable markers such as those described for mammalian cells. In addition, site-specific recombination vectors for easy cloning may also be included. This site-specific recombination regions includes but are not limited to those derived from recombinases such as Flp and Cre and respective binding sites FRT and Lox and modified versions of these (Jensen, Ida 2017). Site-specific recombination may also be achieved using transposases and targeted transposon sequences such as Mu, Tn7, IFP2, piggyback, and engineered versions of these (Wang, Yongjie 2010). Transfection of the expression vector into a host cell can be carried out using standard techniques such as electroporation, nucleofection, calcium-phosphate precipitation, lipofection, polycation-based transfection such as polyethlylenimine (PEI)-based transfection and DEAE-dextran transfection as in mammalian cell expression system.

Suitable insect host cells for transient or constitutive expression of viral vectors, antibodies, antigen binding fragments thereof or variants thereof provided herein include but are not limited to Spodoptera frugiperda derived Sf21 and Sf9, Trichopulsia ni derived Tn5 and High-Five, Drosophila melanogaster derived S2 cells and derivative of these.

In some embodiments, the expression vector is designed such that the expressed protein is secreted into the culture medium in which the host cells are grown. The antibodies, antigen binding fragments thereof or variants thereof can be recovered from the culture medium using standard protein purification methods.

### Purification

Antibodies of the invention or antigen-binding fragments thereof or variants thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragments thereof or variants thereof include naturally purified products, products of chemical synthetic procedures, and products produced by recombinant techniques from an eukaryotic host, including, for example, yeast, higher plant, insect and mammalian cells. Depending upon the host employed in a recombinant production procedure, the antibody of the present invention can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

In preferred embodiments, the antibody is purified (1) to greater than 95% by weight of antibody as determined e.g. by the Lowry method, UV-Vis spectroscopy or by by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or non-reducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

According to one embodiment of the invention, the tissue or liquid sample from the subject is at least one selected from the group consisting of
cardiac tissue,
vasculature,
lung tissue,
renal tissue,
hepatic tissue,
muscle tissue,
skin tissue and/or
blood.

According to yet another one embodiment of the invention, the human or animal subject
suffers from,
is at risk of developing, and/or
is diagnosed for
   a condition selected from the group consisting of a heart, kidney, lung, cardiovascular, cardiorenal and/or cardiopulmonary disease.

According to yet another one embodiment of the invention, the human or animal subject
suffers from,
is at risk of developing, and/or
is diagnosed for
   a condition selected from the group consisting of chronic kidney disease (CKD), diabetic kidney disease (DKD), and heart failure (HF), for example heart failure with preserved ejection fraction (HFpEF).

According to yet another one embodiment of the invention, the human or animal subject comprises an sGC comprising a heme free β1 subunit at least in a particular target tissue. As discussed, said target tissue may be at least one selected from the group consisting of cardiac tissue, vasculature, lung tissue, renal tissue hepatic tissue, muscle tissue, skin tissue and/or blood.

According to another embodiment of the invention, the step of determining whether or not the sample is characterized by the presence, upregulation or overexpression of an sGC comprising a heme free β1 subunit is at least one selected from the group consisting of
ELISA
Immunohistochemistry
Immunoblotting
Immunoprecipitation
Radioimmunoassay, and/or
in situ PCR

ELISA (enzyme-linked immunosorbent assay) is a plate-based assay technique designed for detecting and quantifying substances such as peptides, proteins, antibodies and hormones. Other names, such as enzyme immunoassay (EIA), are also used to describe the same technology.

In Situ Polymerase Chain Reaction (In situ PCR) is a powerful method that detects minute quantities of rare or single-copy number nucleic acid sequences in frozen or paraffin-embedded cells or tissue sections for the localization of those sequences within the cells. The principle of this method involves tissue fixing (to preserve the cell morphology) and subsequent treatment with proteolytic digestion (to provide access for the PCR reagents to the target DNA). The target sequences are amplified by those reagents and then detected by standard immunocytochemical protocols. In situ PCR combines the sensitivity of PCR or RT-PCR amplification along with the ability to perform morphological analysis on the same sample, and thus it is an attractive tool in diagnostic applications

Immunohistochemistry (IHC), sometimes known simply as immunostaining, involves the process of selectively imaging antigens (proteins) in cells of a tissue section by exploiting the principle of antibodies binding specifically to antigens in biological tissues. IHC takes its name from the roots “immuno”, in reference to antibodies used in the procedure, and “histo,” meaning tissue (compare to immunocytochemistry).

Immunoblotting, often referred as Western blot, is a widely used technique to identify specific antigens by antibodies. This involves the identification of a protein target, generally in a complex mixture, via antigen-antibody specific regions. Proteins are typically applied to a gel, separated by electrophoresis according to size, charge, or other differences, and electrophoretically transferred to membranes (usually polyvinylidene difluoride or nitrocellulose). The transferred proteins are bound to the surface of the membrane, providing access for reaction with antibody for detection. All remaining binding sites are blocked by incubating the membrane in a solution containing a protein (casein or bovine serum albumin) or detergent-blocking agents.

After probing with the primary antibody for a specific target the antibody-antigen complexes are visualized through various methods (e.g. fluorescence, chemiluminescence), allowing detection of the specific target protein

Immunoprecipitation is a pull-down assay technique designed for the separation of substances, such as peptides, proteins, nucleic acids, glycans, chemicals and hormones, from a complex mixture. The separation of the target substance (also refered as prey) is mediated by the specific binding of an antibody/immunoglobulin (also referred as capturing antibody or bait) previously coupled to a large particle such as sepharose or agarose beads with or without a magnetic core. Once the target substance is bound to the large particle-antibody complex it can be separated from the complex mixture using physical methods such as centrifugation or magnetic attraction. After stringent washing, the target substance can be eluted from the pull-down beads using extreme pH, high temperatures, high salt concentrations, detergents, orthosteric or allosteric competitors, enzymatic digestion or any other entity or condition disrupting specific antibody binding.

A radioimmunoassay (RIA) is an immunoassay that uses radiolabeled molecules in a stepwise formation of immune complexes. An RIA is a very sensitive in vitro assay technique used to measure concentrations of substances, usually measuring antigen concentrations (for example, hormone levels in blood) by use of antibodies.

According to another aspect of the invention, a monoclonal antibody, or target binding fragment or derivative thereof, or an antibody mimetic or aptamer, is provided, which selectively binds to sGC

According to another embodiment of the invention, the antibody, fragment or derivative which comprises at least one of
a) a set of 3 heavy chain CDRs and 3 light chain CDRs, the set selected from the list according to table 1, and/or
b) a set of 3 heavy chain CDRs and 3 light chain CDRs, the set comprised in the VH and VL sequences of table 2, and/or
c) a heavy chain CDR/light chain CDR combination of a) or b), with the provisio that at least one of the CDRs has up to 3 amino acid substitutions relative to the respective CDR as specified in a) or b), while maintaining its capability to bind to sGC comprising a heme free β1 subunit, and/or
d) a heavy chain CDR/light chain CDR combination of a) or b), with the provisio that at least one of the CDRs has a sequence identity of ≥66% relative to the respective CDR as specified in a) or b), while maintaining its capability to bind to sGC comprising a heme free β1 subunit,
   wherein the CDRs are embedded in a suitable protein framework so as to be capable to bind to sGC comprising a heme free β1 subunit.

As regards option b), it is important to understand that in cases where the VH/VL sequences of an antibody are known, the CDR sequences can be determined with computational methods, like e.g., disclosed in Kunik V, Ashkenazi S and Ofran Y, Nucleic Acids Research, Volume 40, Issue W1, 1 Jul. 2012, Pages W521-W524

TABLE 1
CDR sequences of antibodies disclosed herein

| | set of 3 heavy chain CDRs |
|---|---|
| Antibody name | and 3 light chain CDRs |
| TPP15715 | LCDR1(SEQ ID NO 1) |
| | LCDR2(SEQ ID NO 2) |
| | LCDR3(SEQ ID NO 3) |
| | HCDR1(SEQ ID NO 5) |
| | HCDR2(SEQ ID NO 6) |
| | HCDR3(SEQ ID NO 7), |
| TPP15717 | LCDR1(SEQ ID NO 9) |
| | LCDR2(SEQ ID NO 1) |
| | LCDR3(SEQ ID NO 11) |
| | HCDR1(SEQ ID NO 13) |
| | HCDR2(SEQ ID NO 14) |
| | HCDR3(SEQ ID NO 15) |
| TPP16284 | LCDR1(SEQ ID NO 17) |
| | LCDR2(SEQ ID NO 18) |
| | LCDR3(SEQ ID NO 19) |
| | HCDR1(SEQ ID NO 21) |
| | HCDR2(SEQ ID NO 22) |
| | HCDR3(SEQ ID NO 23) |
| TPP15714 | LCDR1(SEQ ID NO 32) |
| | LCDR2(SEQ ID NO 33) |
| | LCDR3(SEQ ID NO 34) |
| | HCDR1(SEQ ID NO 36) |
| | HCDR2(SEQ ID NO 37) |
| | HCDR3(SEQ ID NO 38) |
| TPP15718 | LCDR1(SEQ ID NO 40) |
| | LCDR2(SEQ ID NO 41) |
| | LCDR3(SEQ ID NO 42) |
| | HCDR1(SEQ ID NO 44) |
| | HCDR2(SEQ ID NO 45) |
| | HCDR3(SEQ ID NO 46) |
| TPP15720 | LCDR1(SEQ ID NO 48) |
| | LCDR2(SEQ ID NO 49) |
| | LCDR3(SEQ ID NO 50) |
| | HCDR1(SEQ ID NO 52) |
| | HCDR2(SEQ ID NO 53) |
| | HCDR3(SEQ ID NO 54) |
| TPP15721 | LCDR1(SEQ ID NO 56) |
| | LCDR2(SEQ ID NO 57) |
| | LCDR3(SEQ ID NO 58) |
| | HCDR1(SEQ ID NO 60) |
| | HCDR2(SEQ ID NO 61) |
| | HCDR3(SEQ ID NO 62) |
| TPP15722 | LCDR1(SEQ ID NO 64) |
| | LCDR2(SEQ ID NO 65) |
| | LCDR3(SEQ ID NO 66) |
| | HCDR1(SEQ ID NO 68) |
| | HCDR2(SEQ ID NO 69) |
| | HCDR3(SEQ ID NO 70) |
| TPP19355 | LCDR1(SEQ ID NO 72) |
| | LCDR2(SEQ ID NO 73) |
| | LCDR3(SEQ ID NO 74) |
| | HCDR1(SEQ ID NO 76) |
| | HCDR2(SEQ ID NO 77) |
| | HCDR3(SEQ ID NO 78) |
| TPP19361 | LCDR1(SEQ ID NO 80) |
| | LCDR2(SEQ ID NO 81) |
| | LCDR3(SEQ ID NO 82) |
| | HCDR1(SEQ ID NO 84) |
| | HCDR2(SEQ ID NO 85) |
| | HCDR3(SEQ ID NO 86) |

TABLE 2
heavy chain/light chain variable domain sequence
pairs of antibodies disclosed herein

| Antibody name | VL/VH Sequences |
|---|---|
| TPP15715 | VL(SEQ ID NO 4) |
| | VH(SEQ ID NO 8) |
| TPP15717 | VL(SEQ ID NO 12 |
| | VH(SEQ ID NO 16) |
| TPP16284 | VL(SEQ ID NO 20) |
| | VH(SEQ ID NO 24) |
| TPP15714 | VL(SEQ ID NO 35) |
| | VH(SEQ ID NO 39) |
| TPP15718 | VL(SEQ ID NO 43) |
| | VH(SEQ ID NO 47) |
| TPP15720 | VL(SEQ ID NO 51) |
| | VH(SEQ ID NO 55) |
| TPP15721 | VL(SEQ ID NO 59) |
| | VH(SEQ ID NO 63) |
| TPP15722 | VL(SEQ ID NO 67) |
| | VH(SEQ ID NO 71) |
| TPP19355 | VL(SEQ ID NO 75) |
| | VH(SEQ ID NO 79) |
| TPP19361 | VL(SEQ ID NO 83) |
| | VH(SEQ ID NO 87) |

TABLE 3
full length light chain/heavy chain sequence
pairs of antibodies disclosed herein

| Antibody name | LC/LH Sequences |
|---|---|
| TPP15715 | LC (SEQ ID NO 26) |
| | HC (SEQ ID NO 27) |
| TPP15717 | LC (SEQ ID NO 28) |
| | HC (SEQ ID NO 29) |
| TPP16284 | LC (SEQ ID NO 30) |
| | HC (SEQ ID NO 31) |
| TPP15714 | VL(SEQ ID NO 88) |
| | VH(SEQ ID NO 89) |
| TPP15718 | VL(SEQ ID NO 90) |
| | VH(SEQ ID NO 91) |
| TPP15720 | VL(SEQ ID NO 92) |
| | VH(SEQ ID NO 93) |
| TPP15721 | VL(SEQ ID NO 94) |
| | VH(SEQ ID NO 95) |
| TPP15722 | VL(SEQ ID NO 96) |
| | VH(SEQ ID NO 97) |
| TPP19355 | VL(SEQ ID NO 98) |
| | VH(SEQ ID NO 99) |
| TPP19361 | VL(SEQ ID NO 100) |
| | VH(SEQ ID NO 101) |

According to a further embodiment of the present invention, preferred antibodies are TPP16284, TPP19355 and TPP19361.

According to a further embodiment of the present invention, preferred antibodies are TPP16284 and TPP19355.

In one embodiment, at least one of the CDRs has a sequence identity of ≥66, preferably ≥67, more preferably any one of ≥68, ≥69, ≥70, ≥71, ≥72, ≥73, ≥74, ≥75, ≥76, ≥77, ≥78, ≥79, ≥80, ≥81, ≥82, ≥83, ≥84, ≥85, ≥86, ≥87, ≥88, ≥89, ≥90, ≥91, ≥92, ≥93, ≥94, ≥95, ≥96, ≥97, ≥98 or most preferably ≥99% sequence identity relative to the respective CDRs. In another embodiment, at least one of the CDRs has been modified by affinity maturation or other modifications, resulting in a sequence modification compared to the sequences disclosed above.

In one embodiment, at least one of the CDRs has up to 2, and preferably 1 amino acid substitutions relative to the respective CDR as specified in a) or b)

According to another embodiment of the invention, the antibody, fragment or derivative comprises
a) a heavy chain/light chain variable domain sequence pair according to table 2
b) the heavy chain/light chain variable domain sequence pair of a), with the provisio that at least one of the sequences thereof has a sequence identity of ≥80% relative to the respective SEQ ID No as shown in table 2, while maintaining its capability to bind to sGC comprising a heme free β1 subunit, and/or
c) the heavy chain/light chain variable domain sequence pair of a), with the provisio that at least one of the sequences thereof has up to 10 amino acid substitutions relative to the respective SEQ ID No as shown in table 2, while maintaining its capability to bind to sGC comprising a heme free β1 subunit.

According to another embodiment of the invention, the antibody, fragment or derivative comprises
a) the full length light chain/heavy chain sequence pair according to table 3
b) the full length light chain/heavy chain sequence pair pair of a), with the provisio that at least one of the sequences thereof has a sequence identity of ≥80% relative to the respective SEQ ID No as shown in table 3, while maintaining its capability to bind to sGC comprising a heme free β1 subunit, and/or
c) the full length light chain/heavy chain sequence pair of a), with the provisio that at least one of the sequences thereof has up to 10 amino acid substitutions relative to the respective SEQ ID No as shown in table 3, while maintaining its capability to bind to sGC comprising a heme free β1 subunit.

In one embodiment, at least one of the sequences has a sequence identity of ≥81, preferably ≥82, more preferably ≥83, ≥84, ≥85, ≥86, ≥87, ≥88, ≥89, ≥90, ≥91, ≥92, ≥93, ≥94, ≥95, ≥96, ≥97, ≥98 or most preferably ≥99% sequence identity relative to the respective SEQ ID No as shown in table 2 or 3.

In one embodiment, at least one of the sequences has up to 9, preferably up to 8, more preferably up to 7, 6, 5, 4, 3 or 2 and most preferably up to 1 amino acid substitutions relative to the respective SEQ ID No as shown in table 2.

According to another embodiment of the invention, at least one amino acid substitution as discussed above is a conservative amino acid substitution. A “conservative amino acid substitution” has a smaller effect on antibody function than a non-conservative substitution. Although there are many ways to classify amino acids, they are often sorted into six main groups on the basis of their structure and the general chemical characteristics of their R groups.

In one embodiment, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Thus, a predicted nonessential amino acid residue in a HR domain polypeptide, for example, is preferably replaced with another amino acid residue from the same side chain family or homologues across families (e.g. asparagine for aspartic acid, glutamine for glutamic acid). Conservative changes can further include substitution of chemically homologous non-natural amino acids (i.e. a synthetic non-natural hydrophobic amino acid in place of leucine, a synthetic non-natural aromatic amino acid in place of tryptophan).

According to another embodiment of the invention, the antibody
has a target binding affinity of ≥50% to sGC comprising a heme free β1 subunit, as measured by SPR compared, to one of the antibodies defined by the sequences as above, and/or
competes for binding to sGC comprising a heme free β1 subunit, with one of the antibodies defined by the sequences as above.

As used herein, the term “competes for binding” is used in reference to one of the antibodies defined by the sequences as above, meaning that the actual antibody as an activity which binds to the same target, or target epitope or domain or subdomain, as does said sequence defined antibody, and is a variant of the latter, or related or dissimilar e. The efficiency (e.g., kinetics or thermodynamics) of binding may be the same as or greater than or less than the efficiency of the latter. For example, the equilibrium binding constant for binding to the substrate may be different for the two antibodies.

According to another embodiment of the invention, the antibody, fragment or derivative, or antibody mimetic or aptamer, is labelled with a detectable marker.

Such detectable marker is for example an enzyme, a luminescent marker, a fluorescent marker, a phosphorescent marker, a radioopaque marker, a radioactive marker, a moiety that can be detected by another binding agent, a marker comprising a nucleotide, or the like. Said marker can be bound to the antibody, fragment or derivative, or antibody mimetic or aptamer, covalently or non-covalently.

According to another aspect of the invention, a companion diagnostic for use in a method according to any the above description is provided, which companion diagnostic comprises a binding molecule which selectively binds to sGC comprising a heme free β1 subunit. A companion diagnostic (CDx) is a diagnostic test or kit used as a companion to a therapeutic drug to determine its applicability to a specific person. Companion diagnostics are often co-developed with drugs to aid in selecting or excluding patient groups for treatment with that particular drug on the basis of their biological characteristics that determine responders and non-responders to the therapy. Companion diagnostics are developed based on companion biomarkers, biomarkers that prospectively help predict likely response or severe toxicity.

According to one embodiment, said binding molecule is an antibody, or fragment or derivative thereof retaining target binding capacity, an antibody mimetic, or an aptamer.

According to another embodiment, said binding molecule is a monoclonal antibody, fragment or derivative thereof, or antibody mimetic or aptamer, as described herein elsewhere.

According to another aspect of the invention, a method for treating a human or animal subject
suffering from,
being at risk of developing, and/or
being diagnosed for
   a condition selected from the group consisting of a heart, kidney, lung, cardiovascular, cardiorenal and/or cardiopulmonary disease is provided, which condition is further characterized by presence, upregulation or overexpression of an sGC comprising a heme free β1 subunit at least in a particular target tissue, with a therapeutically effective amount of an agonist of soluble Guanylyl Cyclase (sGC).

According to another aspect of the invention, a method for treating a human or animal subject
suffering from,
being at risk of developing, and/or
being diagnosed for
   a condition selected from the group consisting of a heart, kidney, lung, cardiovascular, cardiorenal and/or cardiopulmonary disease is provided, which condition is further characterized by presence, upregulation or overexpression of an sGC comprising a heme free β1 subunit at least in a particular target tissue, with a therapeutically effective amount of an activator of soluble Guanylyl Cyclase (sGC).

According to another aspect of the invention, the use of an activator of soluble Guanylyl Cyclase (sGC) (for the manufacture of a medicament) in the treatment of a human or animal subject
suffering from or
being at risk of developing
being diagnosed for,
   a condition selected from the group consisting of a heart, kidney, lung, cardiovascular, cardiorenal and/or cardiopulmonary disease is provided, which condition is further characterized by presence, upregulation or overexpression of an sGC comprising a heme free β1 subunit at least in a particular target tissue.

According to another aspect of the invention, a kit for determining whether a human or animal subject is suitable of being treated with an activator of soluble Guanylyl Cyclase (sGC) is provided, which kit comprises a binding molecule which selectively binds to sGC comprising a heme free β1 subunit.

In one embodiment, said binding molecule is an antibody, or fragment or derivative thereof retaining target binding capacity, or an antibody mimetic, or an aptamer. In another embodiment, said binding molecule is a monoclonal antibody, fragment or derivative as described herein. In another embodiment, said monoclonal antibody, fragment or derivative thereof comprises at least one of the VH/VL pairs from the list disclosed herein, or a modified variant thereof as disclosed herein.

### SEQUENCE LISTING

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones. Note that VH stands for heavy chain, variable domain, and VL stands for light chain, variable domain, and CDR stands for complementarity determining region.

| NO | Qualifier | | Sequence |
|---|---|---|---|
| 1 | TPP15715_VL_5H10 | LCDR1 | TGSSSNIGAGYDVH |
| 2 | TPP15715_VL_5H10 | LCDR2 | ENDRRPS |
| 3 | TPP15715_VL_5H10 | LCDR3 | AAWDDSLNGPL |
| 4 | TPP15715_VL_5H10 | VL | QSVLTQPPSASGTPGQRVTISCTGSSSNIGAGYDVHWY |
| | | | QQLPGTAPKLLIYENDRRPSGVPDRFSGSKSGTSASLA |
| | | | ISGLRSEDEADYYCAAWDDSLNGPLFGGGTKLTVL |
| 5 | TPP15715_VH_5H10 | HCDR1 | NYAMS |
| 6 | TPP15715_VH_5H10 | HCDR2 | AISGSGGSTFYADSVKG |
| 7 | TPP15715_VH_5H10 | HCDR3 | DGTDAFDI |
| 8 | TPP15715_VH_5H10 | VH | EVQLLESGGGLVQPGGSLRLSCAASGFTFSNYAMSWVR |
| | | | QAPGKGLEWVSAISGSGGSTFYADSVKGRFTISRDNSK |
| | | | NTLYLQMNSLRAEDTAVYYCAIDGIDAFDIWGQGTLVT |
| | | | VSS |
| 9 | TPP15717_VL_5D2 | LCDR1 | TGSSSNIGAGYVVH |
| 10 | TPP15717_VL_5D2 | LCDR2 | NNSQRPP |
| 11 | TPP15717_VL_5D2 | LCDR3 | ASWDDSLSGVV |
| 12 | TPP15717_VL_5D2 | VL | QSVLTQPPSASGTPGQRVTISCTGSSSNIGAGYVVHWY |
| | | | QQLPGTAPKLLIYNNSQRPPGVPDRFSGSKSGTSASLA |
| | | | ISGLRSEDEADYYCASWDDSLSGVVFGGGTKLTVL |
| 13 | TPP15717_VH_5D2 | HCDR1 | SYAMS |
| 14 | TPP15717_VH_5D2 | HCDR2 | AISGSGGSTYYADSVKG |
| 15 | TPP15717_VH_5D2 | HCDR3 | EQWLGAEGAFDI |
| 16 | TPP15717_VH_5D2 | VH | EVQLLESGGGLVQPGGSLRLSCAASGFTFSSYAMSWVR |
| | | | QAPGKGLEWVSAISGSGGSTYYADSVKGRFTISRDNSK |
| | | | NTLYLQMNSLRAEDTAVYYCATEQWLGAEGAFDIWGQG |
| | | | TLVTVSS |
| 17 | TPP16284_VL_5C6 | LCDR1 | SGSSNIGNNAVN |
| 18 | TPP16284_VL_5C6 | LCDR2 | GNSNRPS |
| 19 | TPP16284_VL_5C6 | LCDR3 | QSYDSSLSGV |
| 20 | TPP16284_VL_5C6 | VL | QSVLTQPPSASGTPGQRVTISCSGSSSNIGNNAVNWYQ |
| | | | QLPGTAPKLLIYGNSNRPSGVPDRFSGSKSGTSASLAI |
| | | | SGLRSEDEADYYCQSYDSSLSGVFGGGTKLTVL |
| 21 | TPP16284_VH_5C6 | HCDR1 | SYAMS |
| 22 | TPP16284_VH_5C6 | HCDR2 | GVSWNGSRTHYADSVKG |
| 23 | TPP16284_VH_5C6 | HCDR3 | ERLGKWYFDL |
| 24 | TPP16284_VH_5C6 | VH | EVQLLESGGGLVQPGGSLRLSCAASGFTFSSYAMSWVR |
| | | | QAPGKGLEWVSGVSWNGSRTHYADSVKGRFTISRDNSK |
| | | | NTLYLQMNSLRAEDTAVYYCARERLGKWYFDLWGRGIL |
| | | | VTVSS |
| 25 | >sp\|Q02153- | | MYGFVNHALELLVIRNYGPEVWEDIKKEAQLDEEGQFL |
| | 2\|GCYB1 HUMAN Isoform | | VRITYDDSKTYDLVAAASKVLNLNAGEILQMFGKMFFV |
| | HSGC-2 of Guanylate | | FCQESGYDTILRVLGSNVREFLQNLDALHDHLATIYPG |
| | cyclase soluble | | MRAPSFRCTDAEKGKGLILHYYSEREGLQDIVIGIIKT |
| | subunit beta-1 OS = Homo | | VAQQIHGTEIDMKVIQQRNEECDHTQFLIEEKESKEED |
| | sapiens OX = 9606 | | FYEDLDRFEENGTQESRISPYTECKAFPFHTIFDRDLV |
| | GN = GUCY1B1 | | VTQCGNAIYRVLPQLQPGNCSLLSVFSLVRPHIDISFH |
| | | | GILSHINTVFVLRSKEGLLDVEKLECEDELTGTEISCL |
| | | | RLKGQMIYLPEADSILFLCSPSVMNLDDLTRRGLYLSD |
| | | | IPLHDATRDLVLLGEQFREEYKLTQELEILTDRLQLTL |
| | | | RALEDEKKKTDTGIVGFNAFCSKHASGEGAMKIVNLLN |
| | | | DLYTRFDTLTDSRKNPFVYKVETVGDKYMTVSGLPEPC |
| | | | IHHARSICHLALDMMEIAGQVQVDGESVQITIGIHTGE |
| | | | VVIGVIGQRMPRYCLEGNIVNLISRTETTGEKGKINVS |
| | | | EYTYRCLMSPENSDPQFHLEHRGPVSMKGKKEPMQVWF |
| | | | LSRKNTGTEETKQDDD |
| 26 | TPP15715_5H10 full | | QSVLTQPPSASGTPGQRVTISCTGSSSNIGAGYDVHWY |
| | length light chain | | QQLPGTAPKLLIYENDRRPSGVPDRFSGSKSGTSASLA |
| | | | ISGLRSEDEADYYCAAWDDSLNGPLFGGGTKLTVLGQP |
| | | | KAAPSVTLEPPSSEELQANKATLVCLISDFYPGAVIVA |
| | | | WKADSSPVKAGVETTTPSKQSNNKYAASSYLSLTPEQW |
| | | | KSHRSYSCQVTHEGSTVEKTVAPTECS |
| 27 | TPP15715_5H10 full | | EVQLLESGGGLVQPGGSLRLSCAASGFTFSNYAMSWVR |
| | length heavy chain | | QAPGKGLEWVSAISGSGGSTFYADSVKGRFTISRDNSK |
| | | | NTLYLQMNSLRAEDTAVYYCAIDGIDAFDIWGQGTLVT |
| | | | VSSASTKGPSVFPLAPSSKSTSGGTAALGCLVKDYFPE |
| | | | PVTVSWNSGALTSGVHTFPAVLQSSGLYSLSSVVTVPS |
| | | | SSLGTQTYICNVNHKPSNTKVDKKVEPKSCDKTHTCPP |
| | | | CPAPELLGGPSVFLFPPKPKDTLMISRTPEVTCVVVDV |
| | | | SHEDPEVKFNWYVDGVEVHNAKTKPREEQYNSTYRVVS |
| | | | VLTVLHQDWLNGKEYKCKVSNKALPAPIEKTISKAKGQ |
| | | | PREPQVYTLPPSRDELTKNQVSLTCLVKGFYPSDIAVE |
| | | | WESNGQPENNYKTTPPVLDSDGSFFLYSKLTVDKSRWQ |
| | | | QGNVFSCSVMHEALHNHYTQKSLSLSPG |
| 28 | TPP15717 full length | | QSVLTQPPSASGTPGQRVTISCTGSSSNIGAGYVVHWY |
| | light chain | | QQLPGTAPKLLIYNNSQRPPGVPDRFSGSKSGTSASLA |
| | | | ISGLRSEDEADYYCASWDDSLSGVVFGGGTKLTVLGQP |
| | | | KAAPSVTLEPPSSEELQANKATLVCLISDFYPGAVIVA |
| | | | WKADSSPVKAGVETTTPSKQSNNKYAASSYLSLTPEQW |
| | | | KSHRSYSCQVTHEGSTVEKTVAPTECS |
| 29 | TPP15717 full length | | EVQLLESGGGLVQPGGSLRLSCAASGFTFSSYAMSWVR |
| | heavy chain | | QAPGKGLEWVSAISGSGGSTYYADSVKGRFTISRDNSK |
| | | | NTLYLQMNSLRAEDTAVYYCATEQWLGAEGAFDIWGQG |
| | | | TLVTVSSASTKGPSVFPLAPSSKSTSGGTAALGCLVKD |
| | | | YFPEPVTVSWNSGALTSGVHTFPAVLQSSGLYSLSSVV |
| | | | TVPSSSLGTQTYICNVNHKPSNTKVDKKVEPKSCDKTH |
| | | | TCPPCPAPELLGGPSVFLFPPKPKDTLMISRTPEVTCV |
| | | | VVDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSRDELTKNQVSLTCLVKGFYPSD |
| | | | IAVEWESNGQPENNYKTTPPVLDSDGSFFLYSKLTVDK |
| | | | SRWQQGNVFSCSVMHEALHNHYTQKSLSLSPG |
| 30 | TPP16284 full length | | QSVLTQPPSASGTPGQRVTISCSGSSSNIGNNAVNWYQ |
| | light chain | | QLPGTAPKLLIYGNSNRPSGVPDRFSGSKSGTSASLAI |
| | | | SGLRSEDEADYYCQSYDSSLSGVFGGGTKLTVLGQPKA |
| | | | APSVTLFPPSSEELQANKATLVCLISDFYPGAVTVAWK |
| | | | ADSSPVKAGVETTTPSKQSNNKYAASSYLSLTPEQWKS |
| | | | HRSYSCQVTHEGSTVEKTVAPTECS |
| 31 | TPP16284 full length | | EVQLLESGGGLVQPGGSLRLSCAASGFTFSSYAMSWVR |
| | heavy chain | | QAPGKGLEWVSGVSWNGSRTHYADSVKGRFTISRDNSK |
| | | | NTLYLQMNSLRAEDTAVYYCARERLGKWYFDLWGRGIL |
| | | | VTVSSASTKGPSVFPLAPSSKSTSGGTAALGCLVKDYF |
| | | | PEPVTVSWNSGALTSGVHTFPAVLQSSGLYSLSSVVTV |
| | | | PSSSLGTQTYICNVNHKPSNTKVDKKVEPKSCDKTHTC |
| | | | PPCPAPELLGGPSVFLFPPKPKDTLMISRTPEVTCVVV |
| | | | DVSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSRDELTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPG |
| 32 | TPP15714_VL_6B8 | LCDR1 | SGSSSNIGSNYVY |
| 33 | TPP15714_VL_6B8 | LCDR2 | RNNQRPS |
| 34 | TPP15714_VL_6B8 | LCDR3 | TAWDDSLSAVV |
| 35 | TPP15714_VL_6B8 | VL | QSVLTQPPSASGTPGQRVTISCSGSSSNIGSNYVYWYQ |
| | | | QLPGTAPKLLIYRNNQRPSGVPDRFSGSKSGTSASLAI |
| | | | SGLRSEDEADYYCTAWDDSLSAVVFGGGTKLTVL |
| 36 | TPP15714_VH_6B8 | HCDR1 | NYVMS |
| 37 | TPP15714_VH_6B8 | HCDR2 | GVSWNGSRTHYVDSVKR |
| 38 | TPP15714_VH_6B8 | HCDR3 | GLRYSSPFDF |
| 39 | TPP15714_VH_6B8 | VH | EVQLLESGGGLVQPGGSLRLSCAASGFTFSNYVMSWVR |
| | | | QAPGKGLEWVSGVSWNGSRTHYVDSVKRRFTISRDNSK |
| | | | NTLYLQMNSLRAEDTAVYYCARGLRYSSPFDFWG |
| | | | QGTLVTVSS |
| 40 | TPP-15718_VL_5A1 | LCDR1 | SGSSSNIGSNTVN |
| 41 | TPP-15718_VL_5A1 | LCDR2 | GNSNRPS |
| 42 | TPP-15718_VL_5A1 | LCDR3 | AVWDDSLNGWV |
| 43 | TPP-15718_VL_5A1 | VL | QSVLTQPPSASGTPGQRVTISCSGSSSNIGSNTVNWYQ |
| | | | QLPGTAPKLLIYGNSNRPSGVPDRFSGSKSGTSASLAI |
| | | | SGLRSEDEADYYCAVWDDSLNGWVFGGGTKLTVL |
| 44 | TPP-15718_VH_5A1 | HCDR1 | RYGIH |
| 45 | TPP-15718_VH_5A1 | HCDR2 | VISYDGTNKYYADSVKG |
| 46 | TPP-15718_VH_5A1 | HCDR3 | ARSRWASLGAFDI |
| 47 | TPP-15718_VH_5A1 | VH | EVQLLESGGGLVQPGGSLRLSCAASGFTFSRYGIHWVR |
| | | | QAPGKGLEWVAVISYDGTNKYYADSVKGRFTISRDNSK |
| | | | NTLYLQMNSLRAEDTAVYYCARARSRWASLGAFDIWGQ |
| | | | GTLVTVSS |
| 48 | TPP- | LCDR1 | SGSGSNIGNNAVN |
| | 15720_VL_4G10 | | |
| 49 | TPP- | LCDR2 | GNSNRPS |
| | 15720_VL_4G10 | | |
| 50 | TPP- | LCDR3 | QSYGTSLSGSRVL |
| | 15720_VL_4G10 | | |
| 51 | TPP- | VL | QSVLTQPPSASGTPGQRVTISCSGSGSNIGNNAVNWYQ |
| | 15720_VL_4G10 | | QLPGTAPKLLIYGNSNRPSGVPDRFSGSKSGTSASLAI |
| | | | SGLRSEDEADYYCQSYGTSLSGSRVLFGGGTKLTVL |
| 52 | TPP- | HCDR1 | KYWMH |
| | 15720_VH_4G10 | | |
| 53 | TPP- | HCDR2 | SVSASGGSIYYADSVRG |
| | 15720_VH_4G10 | | |
| 54 | TPP- | HCDR3 | GPFWSGYYRLDGLVDY |
| | 15720_VH_4G10 | | |
| 55 | TPP- | VH | EVQLLESGGGLVQPGGSLRLSCAASGFTFRKYWMHWVR |
| | 15720_VH_4G10 | | QTPGKGLEWVSSVSASGGSIYYADSVRGRFTISRDNSK |
| | | | NTLYLQMNSLRAEDTAVYYCARGPFWSGYYRLDGLVDY |
| | | | WGQGTLVTVSS |
| 56 | TPP-15721_VL_4G5 | LCDR1 | SGSSSNIGNNAVN |
| 57 | TPP-15721_VL_4G5 | LCDR2 | RDDRLPS |
| 58 | TPP-15721_VL_4G5 | LCDR3 | SSYTTSSTVV |
| 59 | TPP-15721_VL_4G5 | VL | QSVLTQPPSASGTPGQRVTISCSGSSSNIGNNAVNWYQ |
| | | | QLPGTAPKLLIYRDDRLPSGVPDRFSGSKSGTSASLAI |
| | | | SGLRSEDEADYYCSSYTTSSTVVFGGGTKLTVL |
| 60 | TPP-15721_VH_4G5 | HCDR1 | RYAMS |
| 61 | TPP-15721_VH_4G5 | HCDR2 | GVSWNGSRTHYVGSVKR |
| 62 | TPP-15721_VH_4G5 | HCDR3 | ERLGKWYFDL |
| 63 | TPP-15721_VH_4G5 | VH | EVQLLESGGGLVQPGGSLRLSCAASGFTFSRYAMSWVR |
| | | | QAPGKGLEWVSGVSWNGSRTHYVGSVKRRFTISRDNSK |
| | | | NTLYLQMNSLRAEDTAVYYCARERLGKWYFDLWGQGTL |
| | | | VTVSS |
| 64 | TPP-15722_VL_2A9 | LCDR1 | SGSRSNIGSSVVS |
| 65 | TPP-15722_VL_2A9 | LCDR2 | GNNQRPS |
| 66 | TPP-15722_VL_2A9 | LCDR3 | TSYAGSNNLV |
| 67 | TPP-15722_VL_2A9 | VL | QSVLTQPPSASGTPGQRVTISCSGSRSNIGSSVVSWYQ |
| | | | QLPGTAPKLLIYGNNQRPSGVPDRFSGSKSGTSASLAI |
| | | | SGLRSEDEADYYCTSYAGSNNLVFGGGTKLTVL |
| 68 | TPP-15722_VH_2A9 | HCDR1 | SYSMN |
| 69 | TPP-15722_VH_2A9 | HCDR2 | YISRSSGAIYYADSVKG |
| 70 | TPP-15722_VH_2A9 | HCDR3 | ERLGKWYFDL |
| 71 | TPP-15722_VH_2A9 | VH | EVQLLESGGGLVQPGGSLRLSCAASGFTFSSYSMNWVR |
| | | | QAPGKGLEWVSYISRSSGAIYYADSVKGRFTISRDNSK |
| | | | NTLYLQMNSLRAEDTAVYYCARERLGKWYFDLWGQGTL |
| | | | VTVSS |
| 72 | TPP19355_VL_19C9 | LCDR1 | TGSSSNIGAGYDVH |
| 73 | TPP19355_VL_19C9 | LCDR2 | GNSNRPS |
| 74 | TPP19355_VL_19C9 | LCDR3 | SSYTQNSTRL |
| 75 | TPP19355_VL_19C9 | VL | QSVLTQPPSASGTPGQRVTISCTGSSSNIGAGYDVHWY |
| | | | QQLPGTAPKLLIYGNSNRPSGVPDRFSGSKSGTSASLA |
| | | | ISGLRSEDEADYYCSSYTQNSTRLFGGGTKLTVL |
| 76 | TPP19355_VH_19C9 | HCDR1 | SYSMH |
| 77 | TPP19355_VH_19C9 | HCDR2 | AISGSGGSTYYADSVKG |
| 78 | TPP19355_VH_19C9 | HCDR3 | TPRRWGWSALDY |
| 79 | TPP19355_VH_19C9 | VH | EVQLLESGGGLVQPGGSLRLSCAASGFTFSSYSMHWVR |
| | | | QGPGKGLEWVSAISGSGGSTYYADSVKGRFTISRDNSK |
| | | | NTLYLQMNSLRAEDTAVYYCARTPRRWGWSALDYWGQG |
| | | | TLVTVSS |
| 80 | TPP19361_VL_21C2 | LCDR1 | TGSSSNIGAGYDVH |
| 81 | TPP19361_VL_21C2 | LCDR2 | GNSNRPS |
| 82 | TPP19361_VL_21C2 | LCDR3 | AAWDDSVSGWV |
| 83 | TPP19361_VL_21C2 | VL | QSVLTQPPSASGTPGQRVTISCTGSSSNIGAGYDVHWY |
| | | | QQLPGTAPKLLIYGNSNRPSGVPDRFSGSKSGTSASLA |
| | | | ISGLRSEDEADYYCAAWDDSVSGWVFGGGTKLTVL |
| 84 | TPP19361_VH_21C2 | HCDR1 | SYAMS |
| 85 | TPP19361_VH_21C2 | HCDR2 | AISGSGGSTYYADSVKG |
| 86 | TPP19361_VH_21C2 | HCDR3 | EVWGYSGYDYVDY |
| 87 | TPP19361_VH_21C2 | VH | EVQLLESGGGLVQPGGSLRLSCAASGFTFSSYAMSWVR |
| | | | QAPGKGLEWVSAISGSGGSTYYADSVKGRFTISRDNSK |
| | | | NTLYLQMNSLRAEDTAVYYCAREVWGYSGYDYVDYWGQ |
| | | | GTLVTVSS |

| | | |
|---|---|---|
| 88 | TPP-15714_6B8 full | QSVLTQPPSASGTPGQRVTISCSGSSSNIGSNYVYWYQ |
| | length light chain | QLPGTAPKLLIYRNNQRPSGVPDRFSGSKSGTSASLAI |
| | | SGLRSEDEADYYCTAWDDSLSAVVFGGGTKLTVLGQPK |
| | | AAPSVTLFPPSSEELQANKATLVCLISDFYPGAVTVAW |
| | | KADSSPVKAGVETTTPSKQSNNKYAASSYLSLTPEQWK |
| | | SHRSYSCQVTHEGSTVEKTVAPTECS |
| 89 | TPP-15714_6B8 full | EVQLLESGGGLVQPGGSLRLSCAASGFTFSNYVMSWVR |
| | length heavy chain | QAPGKGLEWVSGVSWNGSRTHYVDSVKRRFTISRDNSK |
| | | NTLYLQMNSLRAEDTAVYYCARGLRYSSPFDFWGQGIL |
| | | VTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKKVEPKSCDKTHTC |
| | | PPCPAPELLGGPSVFLFPPKPKDTLMISRTPEVTCVVV |
| | | DVSHEDPEVKFNWYVDGVEVHNAKTKPREEQYNSTYRV |
| | | VSVLTVLHQDWLNGKEYKCKVSNKALPAPIEKTISKAK |
| | | VGQPREPQVYTLPPSRDELTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPG |
| 90 | TPP-15718_5A1 full | QSVLTQPPSASGTPGQRVTISCSGSSSNIGSNTVNWYQ |
| | length light chain | QLPGTAPKLLIYGNSNRPSGVPDRFSGSKSGTSASLAI |
| | | SGLRSEDEADYYCAVWDDSLNGWVFGGGTKLTVLGQPK |
| | | AAPSVTLFPPSSEELQANKATLVCLISDFYPGAVTVAW |
| | | KADSSPVKAGVETTTPSKQSNNKYAASSYLSLTPEQWK |
| | | SHRSYSCQVTHEGSTVEKTVAPTECS |
| 91 | TPP-15718_5A1 full | EVQLLESGGGLVQPGGSLRLSCAASGFTFSRYGIHWVR |
| | length heavy chain | QAPGKGLEWVAVISYDGTNKYYADSVKGRFTISRDNSK |
| | | NTLYLQMNSLRAEDTAVYYCARARSRWASLGAFDIWGQ |
| | | GTLVTVSSASTKGPSVFPLAPSSKSTSGGTAALGCLVK |
| | | DYFPEPVTVSWNSGALTSGVHTFPAVLQSSGLYSLSSV |
| | | VTVPSSSLGTQTYICNVNHKPSNTKVDKKVEPKSCDKT |
| | | HTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTIS |
| | | KAKGQPREPQVYTLPPSRDELTKNQVSLTCLVKGFYPS |
| | | DIAVEWESNGQPENNYKTTPPVLDSDGSFFLYSKLTVD |
| | | KSRWQQGNVFSCSVMHEALHNHYTQKSLSLSPG |
| 92 | TPP-15720_4G10 full | QSVLTQPPSASGTPGQRVTISCSGSGSNIGNNAVNWYQ |
| | length light chain | QLPGTAPKLLIYGNSNRPSGVPDRFSGSKSGTSASLAI |
| | | SGLRSEDEADYYCQSYGTSLSGSRVLFGGGTKLTVLGQ |
| | | PKAAPSVTLFPPSSEELQANKATLVCLISDFYPGAVTV |
| | | AWKADSSPVKAGVETTTPSKQSNNKYAASSYLSLTPEQ |
| | | WKSHRSYSCQVTHEGSTVEKTVAPTECS |
| 93 | TPP-15720_4G10 full | EVQLLESGGGLVQPGGSLRLSCAASGFTFRKYWMHWVR |
| | length heavy chain | QTPGKGLEWVSSVSASGGSIYYADSVRGRFTISRDNSK |
| | | NTLYLQMNSLRAEDTAVYYCARGPFWSGYYRLDGLVDY |
| | | WGQGTLVTVSSASTKGPSVFPLAPSSKSTSGGTAALGC |
| | | LVKDYFPEPVTVSWNSGALTSGVHTFPAVLQSSGLYSL |
| | | SSVVTVPSSSLGTQTYICNVNHKPSNTKVDKKVEPKSC |
| | | DKTHTCPPCPAPELLGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPAPIEK |
| | | TISKAKGQPREPQVYTLPPSRDELTKNQVSLICLVKGF |
| | | YPSDIAVEWESNGQPENNYKTTPPVLDSDGSFFLYSKL |
| | | TVDKSRWQQGNVFSCSVMHEALHNHYTQKSLSLSPG |
| 94 | TPP-15721_4G5 full | QSVLTQPPSASGTPGQRVTISCSGSSSNIGNNAVNWYQ |
| | length light chain | QLPGTAPKLLIYRDDRLPSGVPDRFSGSKSGTSASLAI |
| | | SGLRSEDEADYYCSSYTTSSTVVFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWKS |
| | | HRSYSCQVTHEGSTVEKTVAPTECS |
| 95 | TPP-15721_4G5 full | EVQLLESGGGLVQPGGSLRLSCAASGFTFSRYAMSWVR |
| | length heavy chain | QAPGKGLEWVSGVSWNGSRTHYVGSVKRRFTISRDNSK |
| | | NTLYLQMNSLRAEDTAVYYCARERLGKWYFDLWGQGTL |
| | | VTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKKVEPKSCDKTHTC |
| | | PPCPAPELLGGPSVFLFPPKPKDTLMISRTPEVTCVVV |
| | | DVSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSRDELTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPG |
| 96 | TPP-15722_2A9 full | QSVLTQPPSASGTPGQRVTISCSGSRSNIGSSVVSWYQ |
| | length light chain | QLPGTAPKLLIYGNNQRPSGVPDRFSGSKSGTSASLAI |
| | | SGLRSEDEADYYCTSYAGSNNLVFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWKS |
| | | HRSYSCQVTHEGSTVEKTVAPTECS |
| 97 | TPP-15722_2A9 full | EVQLLESGGGLVQPGGSLRLSCAASGFTFSSYSMNWVR |
| | length heavy chain | QAPGKGLEWVSYISRSSGAIYYADSVKGRFTISRDNSK |
| | | NTLYLQMNSLRAEDTAVYYCARERLGKWYFDLWGQGTL |
| | | VTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKKVEPKSCDKTHTC |
| | | PPCPAPELLGGPSVFLFPPKPKDTLMISRTPEVTCVVV |
| | | DVSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSRDELTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPG |
| 98 | TPP-19355_19C9 full | QSVLTQPPSASGTPGQRVTISCTGSSSNIGAGYDVHWY |
| | length light chain | QQLPGTAPKLLIYGNSNRPSGVPDRFSGSKSGTSASLA |
| | | ISGLRSEDEADYYCSSYTQNSTRLFGGGTKLTVLGQPK |
| | | AAPSVTLFPPSSEELQANKATLVCLISDFYPGAVTVAW |
| | | KADSSPVKAGVETTIPSKQSNNKYAASSYLSLIPEQWK |
| | | SHRSYSCQVTHEGSTVEKTVAPTECS |
| 99 | TPP-19355_19C9 full | EVQLLESGGGLVQPGGSLRLSCAASGFTFSSYSMHWVR |
| | length heavy chain | QGPGKGLEWVSAISGSGGSTYYADSVKGRFTISRDNSK |
| | | NTLYLQMNSLRAEDTAVYYCARTPRRWGWSALDYWGQG |
| | | TLVTVSSASTKGPSVFPLAPSSKSTSGGTAALGCLVKD |
| | | YFPEPVTVSWNSGALTSGVHTFPAVLQSSGLYSLSSVV |
| | | TVPSSSLGTQTYICNVNHKPSNTKVDKKVEPKSCDKTH |
| | | TCPPCPAPELLGGPSVFLFPPKPKDTLMISRTPEVTCV |
| | | VVDVSHEDPEVKFNWYVDGVEVHNAKTKPREEQYNSTY |
| | | RVVSVLTVLHQDWLNGKEYKCKVSNKALPAPIEKTISK |
| | | AKGQPREPQVYTLPPSRDELTKNQVSLTCLVKGFYPSD |
| | | IAVEWESNGQPENNYKTTPPVLDSDGSFFLYSKLTVDK |
| | | SRWQQGNVFSCSVMHEALHNHYTQKSLSLSPG |
| 100 | TPP-19361_21C2 full | QSVLTQPPSASGTPGQRVTISCTGSSSNIGAGYDVHWY |
| | length light chain | QQLPGTAPKLLIYGNSNRPSGVPDRFSGSKSGTSASLA |
| | | ISGLRSEDEADYYCAAWDDSVSGWVFGGGTKLTVLGQP |
| | | KAAPSVTLEPPSSEELQANKATLVCLISDFYPGAVIVA |
| | | WKADSSPVKAGVETTTPSKQSNNKYAASSYLSLTPEQW |
| | | KSHRSYSCQVTHEGSTVEKTVAPTECS |
| 101 | TPP-19361_21C2 full | EVQLLESGGGLVQPGGSLRLSCAASGFTFSSYAMSWVR |
| | length heavy chain | QAPGKGLEWVSAISGSGGSTYYADSVKGRFTISRDNSK |
| | | NTLYLQMNSLRAEDTAVYYCAREVWGYSGYDYVDYWGQ |
| | | GTLVTVSSASTKGPSVFPLAPSSKSTSGGTAALGCLVK |
| | | DYFPEPVTVSWNSGALTSGVHTFPAVLQSSGLYSLSSV |
| | | VTVPSSSLGTQTYICNVNHKPSNTKVDKKVEPKSCDKT |
| | | HTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTIS |
| | | KAKGQPREPQVYTLPPSRDELTKNQVSLICLVKGFYPS |
| | | DIAVEWESNGQPENNYKTTPPVLDSDGSFFLYSKLTVD |
| | | KSRWQQGNVFSCSVMHEALHNHYTQKSLSLSPG |

### Experiments and Figures

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Any reference signs should not be construed as limiting the scope. All amino acid sequences disclosed herein are shown from N-terminus to C-terminus; all nucleic acid sequences disclosed herein are shown 5′->3′.

### Materials and Methods

### Cell Culture

Spodoptera frugiperda (Sf9) was routinely cultivated in Sf-900 III medium with 1% penicillin/streptomycin at 27° C. 100 rpm. Recombinant rat soluble guanylate cyclase (sGC) proteins were produced in Sf9 cells using the same medium supplemented with 10% fetal calf serum (FCS). For expressing of rat wt-sGC, 0.1 mM of 5-aminolevulinic acid was also added to the culture 30 min before baculovirus infection.

### Baculovirus Stocks

The sequence that codes for rat al subunit was cloned in the pVL1393 fused with a strep-tag sequence (al-StrepII) or a peptide sequence derived from the glycoprotein of the vesicular stomatitis virus (VSV-G) followed by a 6xHis-tag (α1-VSV-His), both at C-terminal of the al subunit. The sequence encoding β1 subunit of rat sGC and the variant with the replacement of the heme ligand, histidine by phenylalanine ((β1-H105F) were also cloned in the pVL1393. In H015F, the substitution of H105 by F105 abolishes the binding of heme, as H105 is very important for heme binding to sGC. The recombinant baculovirus were generated using the FlashBAC Baculovirus Expression System (Oxford Expression Technologies) and after amplification in Sf9 cells, baculovirus stocks were stored at 4° C.

### Production of Recombinant Rat Wt- and Apo-sGC in Sf9 Cells Using Baculovirus Expression Vector System

Sf9 cells grown to a cell density of 5-7×10⁶ cell/mL were diluted in fresh medium to 2×10⁶ cell/mL prior infection. Sf9 cells were co-infected with baculovirus stocks encoding β1HF and α1-StrepII or α1-VSV-His at multiplicity of infection (MOI) 1.5 (0.5α1:1β1HF) to produce rat apo-sGC fused with a Strep tag or rat apo-sGC fused with a VSV-G-His tag at C-terminus, respectively. To produce rat wt-sGC, Sf9 cells were co-infected with baculovirus encoding α1-StrepII and β1 subunits at MOI 4 (2α1:2β1). After 72 h growing at 27° C. 100 rpm, cells were collected by centrifugation at 800×g, 20 min and 4° C. and pellets used for protein isolation.

### Preparation of Cell Extracts Containing Recombinant Rat Wt- and Apo-sGC

Pellets of 2×10⁶ Sf9 cells (expressing rat apo-sGC fused with a Strep tag or a VSV-G-His tag at C-terminus) were ressuspended in 50 mM TAE pH7.6, 0.5 mM EDTA, 7 mM GSH, 0.2 mM PMSF, 1 μM pepstatin A and 1 μM leupeptin at 1.4×10⁷ cell/mL and sonicated 0.6 s at 4° C. Cellular debris were removed by centrifugation at 13000×g, 15 min and 4° C. and supernatants were immediately used for activity assays and phage display.

### Purification of Rat Wt and Apo-sGC

All purification steps were performed at 4° C. After harvesting, cell pellets were ressuspended in lysis buffer (50 mM TAE pH7.4, 1 mM EDTA, 10 mM DTT and 1 tablet anti-proteases cocktail per 50 mL buffer) and homogenized with an Avestin C5 Homogenizer at 600 bar. Homogenate was incubated 30 min at 4° C. with 250 nM avidin and 1 mM PMSF and centrifuged at 30000×g 1 h 30 min and 4° C. The supernatant was filtered and immediately loaded at 1 mL/min in a Tricorn 10/100 containing streptactin superflow high capacity resin previously equilibrated with buffer W (100 mM Tris pH8, 1 M NaCl, 1 mM EDTA, 1 mM benzamidin and 10 mM DTT). After washing column with at least 10 CV, protein was eluted with buffer W supplemented with 2.5 mM desthiobiotin. All fractions contained in the elution peak were pooled and concentrated in a 50 kDa-amicon by successive centrifugations. At this point, rat wt-sGC was further treated with 0.5% Tween20, 20 min at 37° C. to create a heme free version of the wt protein. The last step of purification included a size exclusion chromatography. To this end, concentrated wt- and apo forms of sGC were loaded, separately, onto Superdex 200 16/600 column previous equilibrated with formulation buffer (50 mM TAE pH7.6, 150 mM NaCl, 1 mM EDTA, 10 mM DTT, 1 mM benzamidin and 10% glycerol). Fractions containing dimeric state of the protein were pooled, concentrated in a 50 kDa-amicon, snap-frozen and stored at −80° C. in low protein binding tubes. Purity of the proteins was assessed by SDS-Page and protein concentration determined by the Bradford method.

### Selection of Rat Apo-sGC Binding Molecules by Phage Display

Selection of antibodies targeting rat apo-sGC was performed using BIOINVENT n-CoDeR® Fab Lambda Library. Rat apo-sGC genetically fused with VSV-G epitope tag in the C-terminus of α1 subunit was isolated from the crude extract of 1×10⁷ Sf9 cells using Dynabeads™ M-280 Sheep Anti-Mouse IgG pre-coated with mouse anti-VSV-G monoclonal antibody (P5D4). Alternatively, purified recombinant rat apo-sGC protein fused with a Strep tag at C-terminus was coated to Streptavidin Dynabeads™ M-280. Isolation of rat apo-sGC binding molecules was performed by adding approximately 10′ phage particles from BIOINVENT n-CoDeR® Fab Lambda Library to magnetic Dynabeads covered with rat apo-sGC. After 1 hour incubation at 4° C., unbound phage particles were extensively washed. Magnetic particles with bound phages were used to infect E. coli HB101F′ bacteria strain at exponential growth stage for 30 min at 37° C. enabling phage transfer from magnetic beads to bacteria for infection. Ampicillin resistant bacteria were rescued and used to produce phage particles for subsequent selection rounds. For this strategy three rounds of selection were performed. Rescue of selected phage, removal of bacteriophage gene III fusion and isolation of individual clones was performed using standard methods already described.

Determining Species Apo Selectivity of Selected sFab Reformatted into Full IgG

To determine by ELISA the capacity of individual IgGs to discriminate the apo (heme free) version of wt rat sGC from the heme-loaded version, 5 μg/mL of each individual IgG were coated in Nunc® maxisorp 96-well plates overnight at 4° C. After a minimum 16 hours adsorption period, coated maxisorp plates were blocked with PBS-3% skimmed milk (v/v) prior to the addition of purified rat wt sGC proteins previously treated with 0.5% (v/v) tween to remove the heme group, or not-treated thus preserving heme group. Successful entrapment of heme free or heme-loaded wt rat sGC molecules by reformatted IgG molecules was detected with streptavidin-HRP.

### Determining Apo Selectivity of Selected IgGs in Biological Samples by Western Blot (WB) and Immunohistochemistry (IHC).

To determine the apo selectivity of the obtained antibodies the binding of selected sFab is tested on: a) purified sGC, including WT sGC, oxidized WT sGC (+/−Tween, ODQ) and apo sGC (H105F), on b) cellular extracts from cells overexpressing sGC, including cells overexpressing WT sGC, treated+/−ODQ) and cells overexpressing apo sGC (H105F), on c) cellular extracts from cell lines and primary cells expressing sGC and treated+/−ODQ, on d) tissues and organ homogenates from different species, including mice (e.g. of WT and kiki mice), rats (e.g. WT and RenTG or ZSF-1 rats) and human tissues and comprising but not limited to heart, kidney and lung tissues, and on e) tissue sections from different species including mice (e.g. of WT and kiki mice), rats (e.g. WT and RenTG or ZSF-1 rats) and human tissues, comprising but not limited to heart, kidney and lung tissues.

Read out techniques are western blot (WB) and immunohistochemistry (IHC) of paraffin embedded and cryo-embedded tissue sections performed according to standard laboratory protocols for WBs and IHCs.

For WBs, e.g. denaturing and native gels are used. In addition to selected sFab, negative control ABs, (e.g. TPP-9809) and positive controls for WT sGC (commercial anti-sGC α1 and anti-sGC β1 antibodies) are tested. Assay Conditions include, for the recombinant protein—0.1 ug/lane, the run with 150V 3 h; 4-12% NuPage Bis Tris, MES.

For IHCs specimens are dissected, snap-frozen, OCT-embedded and cryopreserved. After cutting and fixing, slides are washed and stained with 1×PBS buffer, blocking with 5% DKS+0.5% saponing then overnight incubated with the primary antibody and after three time washing (4′ min each) incubated with the secondary antibody for 60 minutes.

Determining Species Cross-Reactivity of Selected sFab

To determine the capacity of individual IgGs to bind different sGC orthologues the mutant H105F apo form of both rat and human sGC was used. Briefly, 5 μg/mL of each individual IgG were coated in Nunc® maxisorp well plates overnight at 4° C. After a minimum 16 hours adsorption period, coated maxisorp plates were blocked with PBS-3% skimmed milk (v/v) prior to the addition of either rat or human sGC mutant proteins. Successful entrapment of rat or human apo-sGC molecules by reformatted IgG molecules was detected with Streptavidin-HRP.

Activation of Recombinant Soluble Guanylate Cyclase (sGC) In Vitro

Investigations on the modulation of recombinant soluble guanylate cyclase (sGC) by the compounds according to the invention with and without sodium nitroprusside, and with and without the heme-dependent sGC inhibitor 1H-1,2,4-oxadiazolo[4,3a]quinoxalin-1-one (ODQ), are carried out by the method described in detail in the following reference: M. Hoenicka, E. M. Becker, H. Apeler, T. Sirichoke, H. Schroeder, R. Gerzer and J.-P. Stasch, “Purified soluble guanylyl cyclase expressed in a baculovirus/Sf9 system: Stimulation by YC-1, nitric oxide, and carbon oxide”, J. Mol. Med. 77 (1999), 14-23. The heme free guanylate cyclase is obtained by adding Tween 20 to the sample buffer (0.5% in the final concentration).

As described in WO 2012/139888, combination of sGC activators and 2-(N,N-diethylamino)-diazenolate 2-oxide (DEA/NO), an NO donor, show no synergistic effect, i.e. the effect of DEA/NO is not potentiated as is expected with an sGC modulator acting via a heme-dependent mechanism. In addition, the effect of the sGC activator according to the invention is not blocked by 1H-1,2,4-oxadiazolo[4,3a]quinoxalin-1-one (ODQ), a heme-dependent inhibitor of soluble guanylate cyclase, but is in fact increased. Thus, this test is suitable to distinguish between the heme-dependent sGC Stimulators and the heme-independent sGC Activators.

### FIGURES

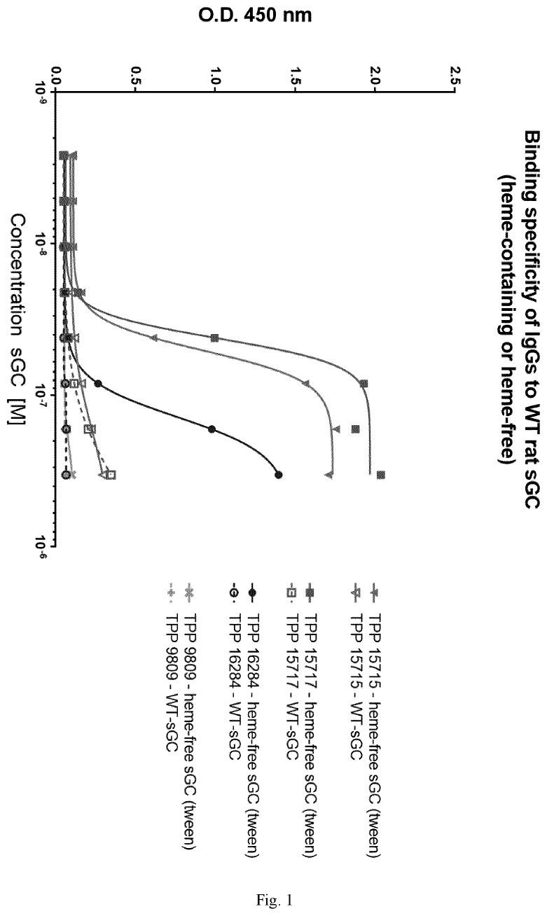

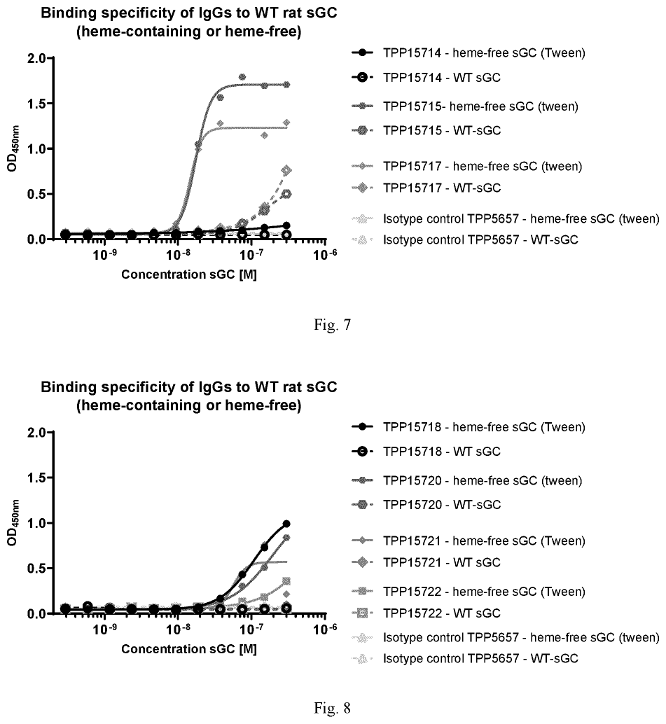

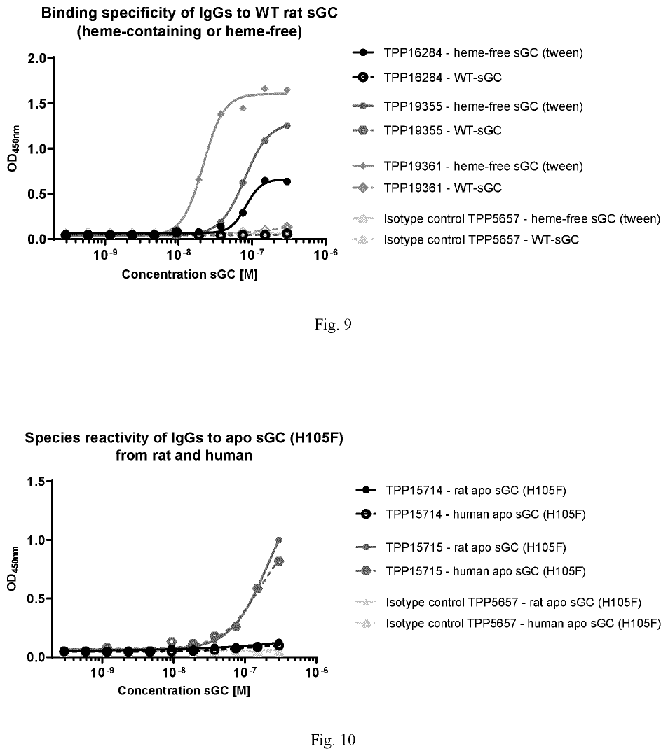

FIG. 1 and FIGS. 7-9 show the results of the antibodies obtained in the described lead discovery process as described in the experimental section above, by ELISA. Ten antibodies (TPP15715, TPP15717, TPP16284, TPP15714, TPP15718, TPP15720, TPP15721, TPP15722, TPP19355 and TPP19361) could be determined which have nM affinity for heme free sGC (as obtained by tween treatment), while the isotype control (TPP9809 and TPP5657) did not bind. TPP15715, TPP15717 and TPP16284 were then further profiled. The affinities determined by SPR were as follows:

| | K_{D} | |
|---|---|---|
| | (WT rat sGC heme free | K_{D} |
| | (tween treated)) | (WT rat sGC heme-loaded) |
| TPP15715 | 50 nM | No binding |
| TPP15717 | 42 nM | No binding |
| TPP16284 | 139 nM | No binding |
| TPP9809 | No binding | No binding |
| (Isotype Control) | | |

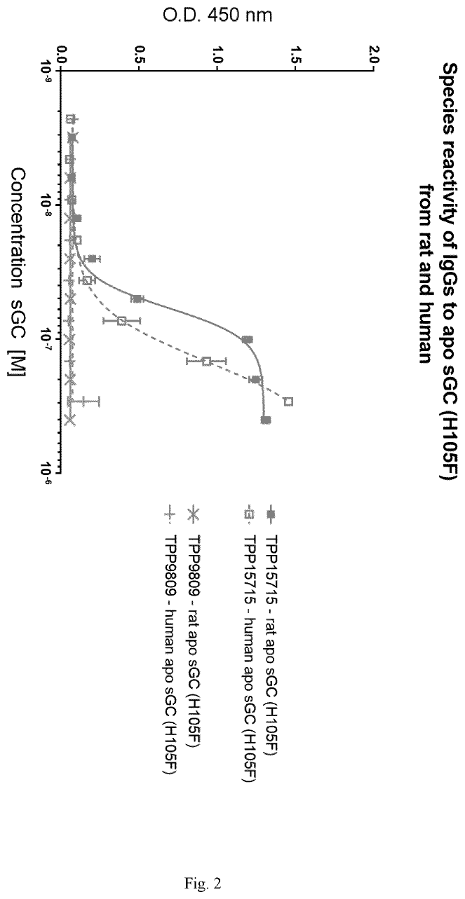

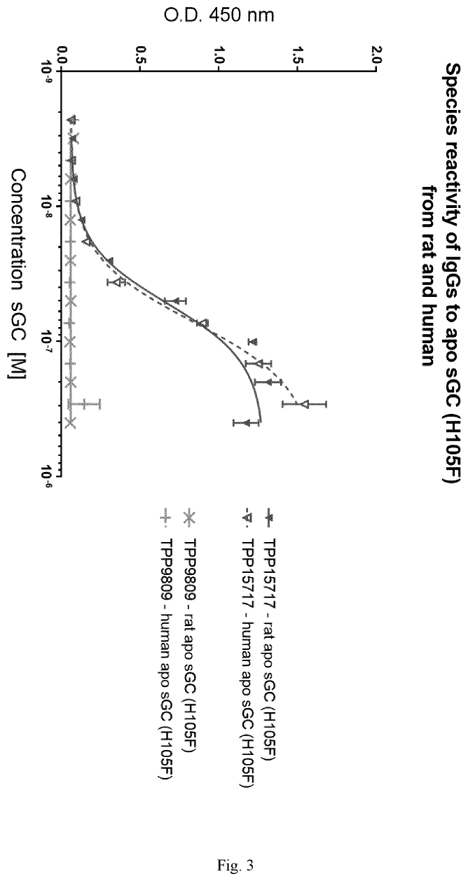

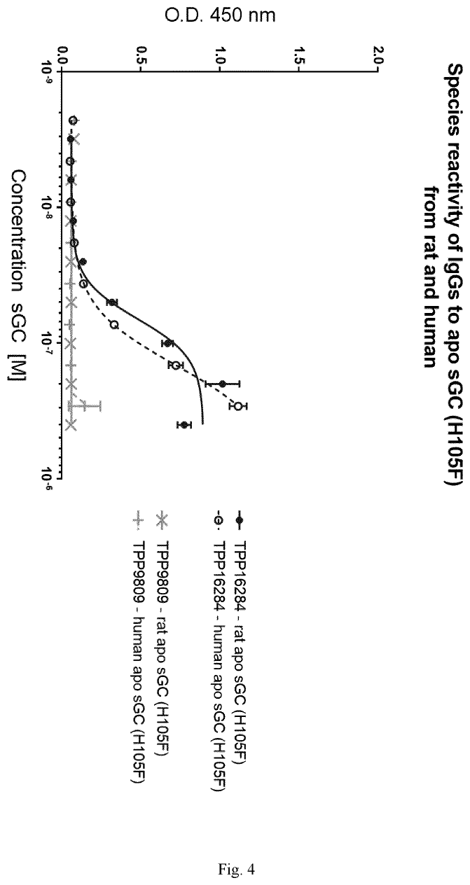

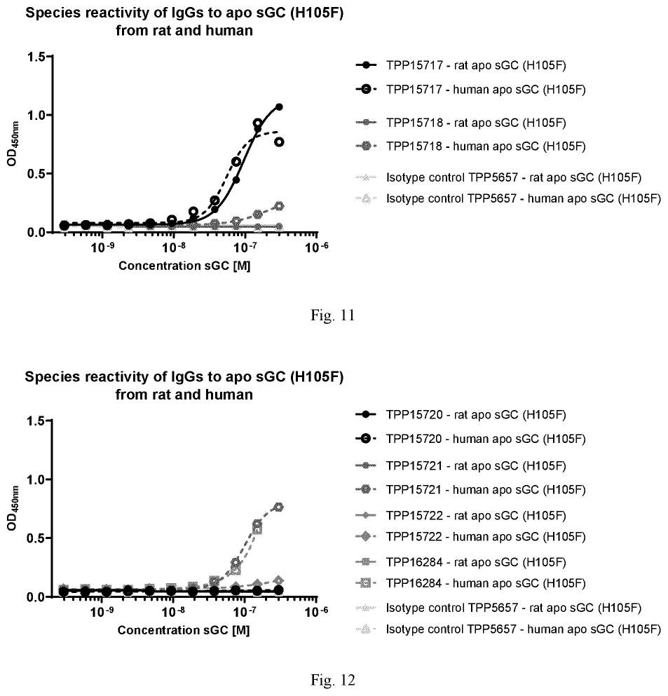

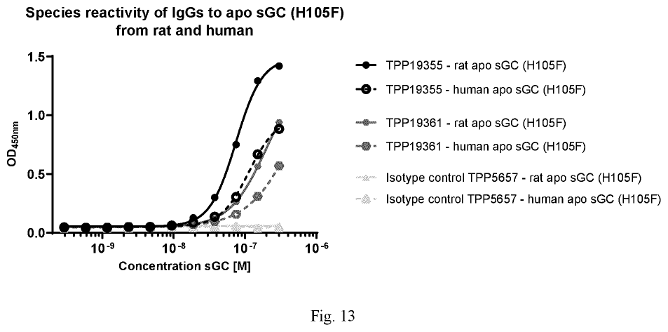

FIGS. 2-4 and FIGS. 10-13 show the species reactivity of the ten respective IgGs to apo-sGC (H105F) from rat and human. It was found that the ten selected antibodies bind both rat and human heme free sGC. TPP15715, TPP15717 and TPP16284 were then further profiled. The affinities determined by SPR were as follows:

| | K_{D} | K_{D} |
|---|---|---|
| | (rat apo-sGC (H105F)) | (Human apo-sGC (H105F)) |
| TPP15715 | 59 nM | 148 nM |
| TPP15717 | 45 nM | 72 nM |
| TPP16284 | 67 nM | 146 nM |
| TPP9809 | No binding | No binding |
| (Isotype Control) | | |

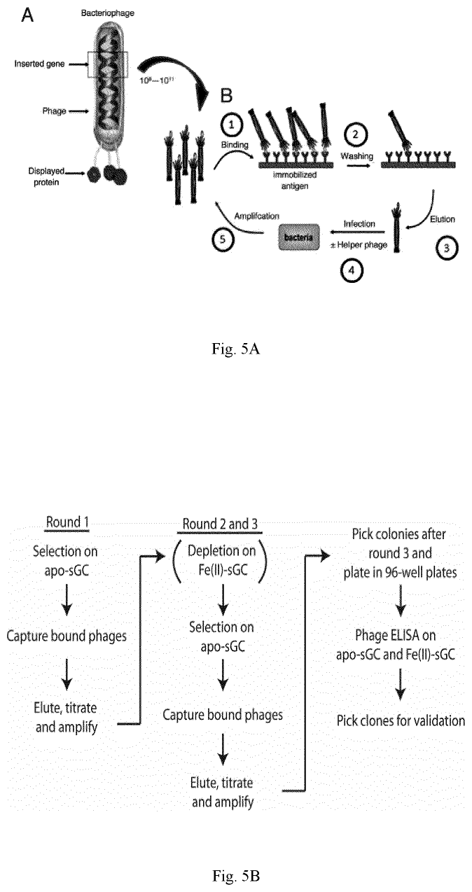

FIGS. 5A and B show the details of the antibody screening process.

### REFERENCES

Bunch et al., Nucleic Acids Res. (1988) Feb. 11; 16(3):1043-61
Chung et al., Mol Cell Biol. (1990) Dec. 10(12):6172-80
Evgenov et al., Nat Rev Drug Discov. (2006) September; 5(9):755-68.
Farrell et al., Biotechnol Bioeng. (1998) Dec. 20; 60(6):656-63
Follmann et al., J. Med Chem (2017) June; 22; 60(12):5146-5161
Hoenicka et al., (1999) J Mol Med January; 77(1):14-23
Hoet et al., Nature Biotechnology (2005) March; 23(3), 344-348
Jensen et al., Protein J. (2017) August; 36(4):332-342
Kunik et al., Nucleic Acids Res. (2012), 40:W521-524
Ren et al., Afr J Biotechnol. (2011) 10(44):8930-8941
Stasch et al., Nature (2001) Mar. 8; 410(6825):212-5
Stasch et al., Br. J. Pharmacol. July; 136 (2002), 773-783
Stasch et al., J. Clin. Invest. September; 116 (2006), 2552-2561
Stasch & Hobbs, Handb Exp Pharmacol. (2009); 191:277-308
Wang et al., J Virol Methods (2010) July; 167(1):95-9
Wu et al., J Biotechnol. (2000) June 9; 80(1):75-83

1. A method for determining whether a human or animal subject
suffers from oxidative stress
is suitable of being treated with an antioxidant and/or free radical scavenger, and/or
is suitable of being treated with an activator of sGC
said method comprising the step of
determining whether or not a tissue or liquid sample from said subject is characterized by the presence, upregulation or overexpression of sGC comprising a heme free β1 subunit.

2. The method according to claim 1, wherein said activator of soluble Guanylyl Cyclase (sGC), is at least one selected from the group consisting of
4-({(4-carboxybutyl)[2-(2-{[4-(2-phenylethyl)benzyl]oxy}phenyl)ethyl]amino}methyl)benzoic acid
5-chloro-2-(5-chlorothiophene-2-sulfonylamino-N-(4-(morpholine-4-sulfonyl)phenyl)benzamide as sodium salt
2-(4-chlorophenylsulfonylamino)-4,5-dimethoxy-N-(4-(thiomorpholine-4-sulfonyl)phenyl)benzamide
1-{6-[5-chloro-2-({4-trans-4-}trifluoromethyl)cyclohexyl]benzyl}oxy)phenyl]pyridin-2-yl}-5-(trifluoromethyl)-1H-pyrazole-4-carboxylic acid
1-[6-(2-(2-methyl-4-(4-trifluoromethoxyphenyl)benzyloxy)phenyl)pyridin-2-yl]-5-trifluoromethylpyrazole-4-carboxylic acid
1[6-(3,4-dichlorophenyl)-2-pyridinyl-5-(trifluoromethyl)-1H-pyrazole-4-carboxylic acid
1-({2-[3-chloro-5-(trifluoromethyl)phenyl]-5-methyl-1,3-thiazol-4-yl}methyl)-1H-pyrazole-4-carboxylic acid
4-({2-[3-(trifluoromethyl)phenyl]-1,3-thiazol-4-yl}methyl)benzoic acid
1-({2-[2-fluoro-3-(trifluoromethyl)phenyl]-5-methyl-1,3-thiazol-4-yl}methyl)-1H-pyrazole-4-carboxylic acid
3-(4-chloro-3-{[(2S,3R)-2-(4-chlorophenyl)-4,4,4-trifluoro-3-methylbutanoyl]amino}phenyl)-3-cyclopropylpropanoic acid
5-{[2-(4-carboxyphenyl)ethyl][2-(2-{[3-chloro-4′-(trifluoromethyl)biphenyl-4-yl]methoxy}phenyl)ethyl]amino}-5,6,7,8-tetrahydroquinoline-2-carboxylic acid formula
5-{(4-carboxybutyl)[2-(2-{[3-chloro-4′-(trifluoromethyl)biphenyl-4-yl]methoxy}phenyl)ethyl]amino}-5,6,7,8-tetrahydroquinoline-2-carboxylic acid of the formula
(1R,5S)-3-[4-(5-methyl-2-{[2-methyl-4-(piperidin-1-ylcarbonyl)benzyl]oxy}phenyl)-1,3-thiazol-2-yl]-3-azabicyclo[3.2.1]octane-8-carboxylic acid
1-[6-(5-methyl-2-{[2-(tetrahydro-2H-pyran-4-yl)-1,2,3,4-tetrahydroisoquinolin-6-yl]methoxy}phenyl)pyridin-2-yl]-5-(trifluoromethyl)-1H-pyrazole-4-carboxylic acid
4-[[(4-Carboxybutyl)[2-[2-[[4-(2-phenylethyl)phenyl]methoxy]phenyl]ethyl]amino]methyl]benzoic acid
BAY 60-2770 4-({(4-carboxybutyl) [2-(5-fluoro-2-{[40-(trifluoromethyl) biphenyl-4-yl]methoxy}phenyl)ethyl] amino}methyl)benzoic acid) and
(S)-1-(6-(3-((4-(1-(cyclopropanecarbonyl)piperidin-4-yl)-2-methylphenyl)amino)-2,3-dihydro-1H-inden-4-yl)pyridin-2-yl)-5-methyl-1H-pyrazole-4-carboxylic acid

3. The method according to claim 1, wherein in the step for determining whether or not said sample is characterized by the presence, upregulation or overexpression of sGC comprising a heme free β1 subunit, a binding molecule is used which selectively binds to sGC comprising a heme free β1 subunit.

4. The method according to claim 3, wherein said binding molecule is an antibody, or fragment or derivative thereof retaining target binding capacity, an antibody mimetic, or an aptamer.

5. The method according to claim 1, wherein the tissue or liquid sample from the subject is at least one selected from the group consisting of
cardiac tissue,
vasculature,
lung tissue,
renal tissue,
hepatic tissue,
muscle tissue,
skin tissue and/or
blood.

6. The method according to claim 1, wherein the human or animal subject
suffers from,
is at risk of developing and/or
is diagnosed for
a condition selected from the group consisting of a heart, kidney, lung, cardiovascular, cardiorenal and/or cardiopulmonary disease.

7. A monoclonal antibody, or target binding fragment or derivative thereof, or an antibody mimetic or aptamer, which selectively binds to sGC comprising a heme free β1 subunit.

8. The antibody, fragment or derivative according to claim 7, which comprises at least one of
a) a set of 3 heavy chain CDRs and 3 light chain CDRs, the set selected from the list according to table 1, and/or
b) a set of 3 heavy chain CDRs and 3 light chain CDRs, the set comprised in the VH and VL sequences of table 2, and/or
c) a heavy chain CDR/light chain CDR combination of a) or b), with the provisio that at least one of the CDRs has up to 3 amino acid substitutions relative to the respective CDR as specified in a) or b), while maintaining its capability to bind to sGC comprising a heme free β1 subunit, and/or
d) a heavy chain CDR/light chain CDR combination of a) or b), with the provisio that at least one of the CDRs has a sequence identity of ≥66% relative to the respective CDR as specified in a) or b), while maintaining its capability to bind to sGC comprising a heme free β1 subunit,
wherein the CDRs are embedded in a suitable protein framework so as to be capable to bind to sGC comprising a heme free β1 subunit.

9. The antibody, fragment or derivative according to claim 7, which comprises
a) a heavy chain/light chain variable domain sequence pair according to table 2
b) the heavy chain/light chain variable domain sequence pair of a), with the provisio that at least one of the sequences thereof has a sequence identity of ≥80% relative to the respective SEQ ID No as shown in table 2, while maintaining its capability to bind to sGC comprising a heme free β1 subunit, and/or
c) the heavy chain/light chain variable domain sequence pair of a), with the provisio that at least one of the sequences thereof has up to 10 amino acid substitutions relative to the respective SEQ ID No as shown in table 2, while maintaining its capability to bind to sGC comprising a heme free β1 subunit.

10. A companion diagnostic for use in a method according to claim 1, which companion diagnostic comprises a binding molecule which selectively binds to sGC comprising a heme free β1 subunit.

11. The companion diagnostic according to claim 10, wherein said binding molecule is a monoclonal antibody, fragment or derivative thereof which selectively binds to sGC comprising a heme free β1 subunit.

12. A method for treating a human or animal subject
suffering from,
being at risk of developing, and/or
being diagnosed for
a condition selected from the group consisting of a heart, kidney, lung, cardiovascular, cardiorenal and/or cardiopulmonary disease, which condition is further characterized by presence, upregulation or overexpression of an sGC comprising a heme free β1 subunit at least in a particular target tissue, said method comprising administering a therapeutically effective amount of an activator of soluble Guanylyl Cyclase (sGC) to the human or animal subject in need thereof.

13. (canceled)

14. (canceled)

15. A kit for determining whether a human or animal subject is suitable of being treated with an activator of soluble Guanylyl Cyclase (sGC), which kit comprises a binding molecule which selectively binds to sGC comprising a heme free β1 subunit.
